(19) United States
(12) Patent Application Publication
(10) Pub. No.: US 2011/0124877 A1
(43) Pub. Date: May 26, 2011

(54) 5-BENZYL-4-AZOLYLMETHYL-4-SPIRO[2.4]HEPTANOL DERIVATIVES, METHODS FOR PRODUCING THE SAME, AND AGRO-HORTICULTURAL AGENTS AND INDUSTRIAL MATERIAL PROTECTING AGENTS THEREOF

(75) Inventors: Atsushi Ito, Tokyo (JP); Takashi Shimokawara, Tokyo (JP); Eiyu Imai, Tokyo (JP); Yoichi Kanda, Tokyo (JP); Nobuyuki Kusano, Tokyo (JP); Masaru Mori, Tokyo (JP); Rumi Suzuki, Tokyo (JP)

(73) Assignee: KUREHA CORPORATION, Tokyo (JP)

(21) Appl. No.: 12/999,822

(22) PCT Filed: Aug. 24, 2009
(86) PCT No.: PCT/JP2009/004053

§ 371 Date: Jan. 28, 2011

(30) Foreign Application Priority Data
Aug. 26, 2008 (JP) 2008-216475

(51) Int. Cl. C07D 249/08; C07D 233/60; C07D 303/08; C07D 303/22; C07D 303/38; C07C 49/697; C07C 49/67; C07C 49/753; C07C 255/56; C07C 69/757

(52) U.S. Cl. 548/267.4; 548/267.8; 548/300.7; 549/332; 568/327; 558/384; 568/330; 558/414; 560/53

(57) ABSTRACT

To provide a novel 5-benzyl-4-azolylmethyl-4-spiro[2.4]heptanol derivative, a method for producing the same, and an agro-horticultural agent and an industrial material protecting agent containing as an active ingredient the abovementioned 5-benzyl-4-azolylmethyl-4-spiro[2.4]heptanol derivative.

To be a 5-benzyl-4-azolylmethyl-4-spiro[2.4]heptanol derivative represented by Formula (I):

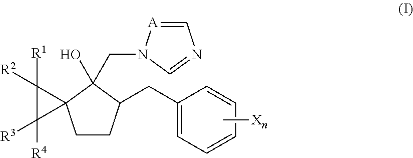

wherein X denotes a halogen atom, a C₁-C₅ alkyl group, a C₁-C₅ haloalkyl group, a C₁-C₅ alkoxy group, a C₁-C₅haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and A denotes a nitrogen atom or a methyne group.

### TECHNICAL FIELD

The present invention relates to a 5-benzyl-4-azolylmethyl-4-spiro[2.4]heptanol derivative, a method for producing the same, and an agro-horticultural agent and an industrial material protecting agent containing such a 5-benzyl-4-azolylmethyl-4-spiro[2.4]heptanol derivative as an active ingredient.

### BACKGROUND ART

Conventionally, a novel fungicidal compound has been desired in view of prevention of environmental pollution or drug resistances. For example, a large number of products, especially fungicides, containing a triazole groups are known. Triazole fungicides containing cyclopentane rings are also known, and disclosed for example in Patent Literatures 1 to 4. In addition, Triazole fungicides containing cycloalkyl groups are also known, and disclosed for example in Patent Literatures 5 and 6.

Certain triazole or imidazole derivatives containing cyclopropyl groups are disclosed for example in Patent Literatures 7 to 13.

Also in Patent Literature 14, certain triazole or imidazole derivatives containing spiro rings are disclosed.

Compounds described in these references have structures different from that of a 5-benzyl-4-azolylmethyl-4-spiro[2.4]heptanol derivative according to the invention.

### CITATION LIST

### Patent Literature

[PTL 1] JP A 63-156782 (corresponding to EP0272895, ES2030080 etc)
[PTL 2] JP A 1-93574 (corresponding to AR245703, EP0267778, ES2053564 etc)
[PTL 3] JP A 2-237979 (corresponding to EP0378953, ES2087873, AU4734889 etc)
[PTL 4] JP A 62-149667 (corresponding to ES2006179, GB2180236 etc)
[PTL 5] JP A 1-186869 (corresponding to EP0324646, ES2055026 etc)
[PTL 6] JP A 60-215674 (corresponding to EP0153797 etc)
[PTL 7] JP A 56-97276 (corresponding to ES8204428, GB2064520 etc)
[PTL 8] JP A 61-126049 (corresponding to EP0180136, ES8701732 etc)
[PTL 9] JP A 2-286664 (corresponding to CA2011085, EP0390022 etc)
[PTL 10] EP0047594 (corresponding to JP A 55-122771 etc)
[PTL 11] EP0052424 (corresponding to JP A 57-114577 etc)
[PTL 12] EP0212605 (corresponding to ES2001270, JP A 62-51670 etc)
[PTL 13] JP A 11-80126
[PTL 14] JP A 7-285943 (corresponding to CA2093623, EP0565463 etc)
[PTL 15] JP A 1-301664 (corresponding to EP0329397 etc) (will after be described in paragraph 0083)

### SUMMARY OF INVENTION

### Technical Problem

Conventionally, an agro-horticultural pesticide having a low toxicity to humans, capable of being handled safely, and exhibiting an excellent inhibitory effect on a wide range of plant diseases has been desired. Also, there has been a need for a plant growth regulator which regulates the growth of a variety of crops and horticultural plants whereby exhibiting yield-increasing effects or quality-improving effects, as well as an industrial material protecting agent which protects an industrial material from a wide range of hazardous microorganisms which invades such materials.

Accordingly, the present invention aims primarily at providing a novel 5-benzyl-4-azolylmethyl-4-spiro[2.4]heptanol derivative exhibiting an excellent agro-horticultural disease controlling effect, a plant growth regulating effect and an industrial material protecting effect, a method for producing the same, and an agro-horticultural agent and an industrial material protecting agent containing the aforementioned 5-benzyl-4-azolylmethyl-4-spiro[2.4]heptanol derivative as an active ingredient.

### Solution to Problem

To achieve the aim mentioned above, the invention first provides a 5-benzyl-4-azolylmethyl-4-spiro[2.4]heptanol derivative represented by Formula (I).

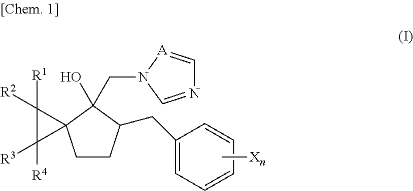

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and A denotes a nitrogen atom or a methyne group.

The invention also provides a method for producing a 5-benzyl-4-azolylmethyl-4-spiro[2.4]heptanol derivative represented by Formula (I) comprising reacting an oxirane derivative represented by Formula (II), which is obtained by oxiranylating a carbonyl compound represented by Formula (IV), with a 1,2,4-triazole or imidazole compound represented by Formula (III).

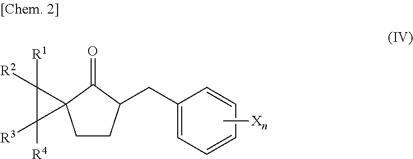

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group.

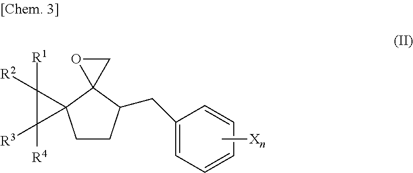

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group.

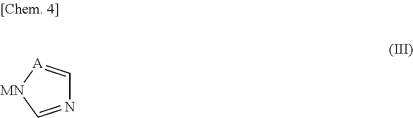

wherein M denotes a hydrogen atom or an alkaline metal; and A denotes a nitrogen atom or a methyne group.

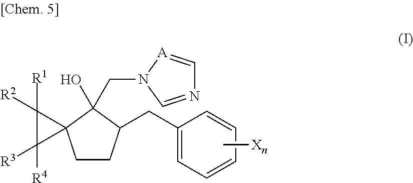

wherein X, N, R¹, R², R³ and R⁴ correspond to the X, N, R¹, R², R³ and R⁴ as defined in Formula II described above; and A corresponds to the A as defined in Formula III described above.

Although it is possible here that an oxirane derivative represented by Formula (II) which is obtained by oxiranylating a carbonyl compound represented by Formula (IV), with a 1,2,4-triazole or imidazole compound represented by Formula (III) is first produced and subsequently reacted with a 1,2,4-triazole or imidazole compound represented by Formula (III), a method in which upon the oxiranylation the 1,2,4-triazole or imidazole compound represented by Formula (III) is allowed to coexist and the carbonyl compound represented by Formula (IV) is oxiranylated while reacting the 1,2,4-triazole or imidazole compound represented by Formula (III) whereby producing a 5-benzyl-4-azolylmethyl-4-spiro[2.4]heptanol derivative represented by Formula (I) is also included.

Furthermore, the invention provides an agro-horticultural pesticide containing a 5-benzyl-4-azolylmethyl-4-spiro[2.4]heptanol derivative represented by Formula (I).

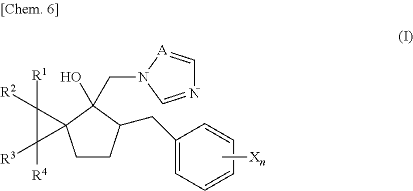

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and A denotes a nitrogen atom or a methyne group.

### Advantageous Effects of Invention

According to the invention a novel 5-benzyl-4-azolylmethyl-4-spiro[2.4]heptanol derivative exhibiting excellent agro-horticultural fungicidal effect, plant growth regulating effect, and industrial material protecting effect, a method for producing the same, and an agro-horticultural agent and an industrial material protecting agent containing a 5-benzyl-4-azolylmethyl-4-spiro[2.4]heptanol derivative described above as an active ingredient.

### DESCRIPTION OF EMBODIMENTS

Preferred embodiments of the invention are described below while referring to Figures. The following embodiments only exemplify the representatives of the invention and do not restrict the scope of the invention.

### A) 5-Benzyl-4-azolylmethyl-4-spiro[2.4]heptanol derivative

A 5-benzyl-4-azolylmethyl-4-spiro[2.4]heptanol derivative according to the invention is represented by Formula (I) shown above. The followings are the details of the 5-benzyl-4-azolylmethyl-4-spiro[2.4]heptanol derivative according to the invention.

In the chemical formula (I) shown above, the X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group. As used herein, the halogen atom may for example be a chlorine atom, a fluorine atom, a bromine atom, and an iodine atom. The C₁-C₅ alkyl group may for example be a methyl group, an ethyl group, an n-propyl group, an i-propyl group, an n-butyl group, an i-butyl group, a s-butyl group, a t-butyl group and the like. The C₁-C₅ haloalkyl group may for example be a trifluoromethyl group, a 1,1,2,2,2-pentafluoroethyl group, a chloromethyl group, a trichloromethyl group, a bromomethyl group and the like. The C₁-C₅ alkoxy group may for example be a methoxy group, an ethoxy group, an n-propoxy group and the like. The C₁-C₅ haloalkoxy group may for example be a trifluoromethoxy group, a difluoromethoxy group, a 1,1,2,2,2-pentafluoroethoxy group, a 2,2,2-trifluoroethoxy group and the like.

Among the above definitions, the following substituents Xs are more preferred; fluorine atom, chlorine atom, bromine atom, iodine atom, methyl group, trifluoromethyl group, difluoromethoxy group, trifluomethoxy group, methoxy group or phenyl group.

Among the above definitions, the following substituents Xs are still more preferred; fluorine atom, chlorine atom, bromine atom or trifluoromethyl group.

The n is an integer of 0 to 5. When n is not less than 2, Xs may be the same or different. The n is preferably in the range of 1 to 2. It is still more preferred that n is 1 and X is bonded to the 4-position.

The R¹, R², R³, R⁴ each independently denotes a hydrogen atom, a halogen atom or a C₁-C₅ alkyl group. As used herein, the halogen atom may for example be a chlorine atom, a fluorine atom, a bromine atom, and an iodine atom. The C₁-C₅ alkyl group may for example be a methyl group, an ethyl group, an n-propyl group, an i-propyl group, an n-butyl group, an i-butyl group, a s-butyl group, a t-butyl group and the like. Among the above definitions, the following substituents R¹, R², R³, R⁴ are more preferred; hydrogen atom, methyl group or chlorine atom. The following substituents R¹, R², R³, R⁴ are still more preferred; hydrogen atom or methyl group.

The A denotes a nitrogen atom or a methyne group. A nitrogen atom is more preferred.

Depending on the combination of types of the substituents for the X, R¹, R², R³, R⁴ and A and numerical values for the n described above, the compounds shown in Tables 1 to 20 are exemplified as the 5-benzyl-4-azolylmethyl-4-spiro[2.4]heptanol derivatives according to the invention.

While a 5-benzyl-4-azolylmethyl-4-spiro[2.4]heptanol derivative according to the invention may exist as any of the stereoisomers (C-form and T-form) represented by Formula (I-C) and Formula (I-T) shown below, either isomer as well as their mixture may be employed. With regard to the chemical formulae shown below, the relative configuration of one whose hydroxyl group in the 4-position and benzyl group in the 5-position are in a cis relationship is designated as (I-C), and the relative configuration of one whose relevant groups are in a trans relationship is designated as (I-T).

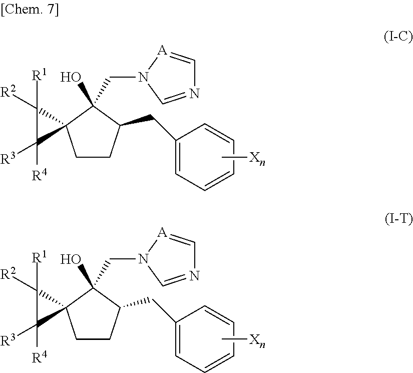

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and A denotes a nitrogen atom or a methyne group.

TABLE 1

| Compound No | R¹ | R² | R³ | R⁴ | Xn^{)} | A | Isomer type |
|---|---|---|---|---|---|---|---|
| I-1 | H | H | H | H | 4-Cl | N | C |
| I-2 | H | H | H | H | 4-Cl | N | T |
| I-3 | H | H | H | H | 3-Cl | N | C |
| I-4 | H | H | H | H | 3-Cl | N | T |
| I-5 | H | H | H | H | 2-Cl | N | C |
| I-6 | H | H | H | H | 2-Cl | N | T |
| I-7 | H | H | H | H | 4-F | N | C |
| I-8 | H | H | H | H | 4-F | N | T |
| I-9 | H | H | H | H | 3-F | N | C |
| I-10 | H | H | H | H | 3-F | N | T |
| I-11 | H | H | H | H | 2-F | N | C |
| I-12 | H | H | H | H | 2-F | N | T |
| I-13 | H | H | H | H | 4-Br | N | C |
| I-14 | H | H | H | H | 4-Br | N | T |
| I-15 | H | H | H | H | 3-Br | N | C |
| I-16 | H | H | H | H | 3-Br | N | T |
| I-17 | H | H | H | H | 2-Br | N | C |
| I-18 | H | H | H | H | 2-Br | N | T |
| I-19 | H | H | H | H | 4-I | N | C |
| I-20 | H | H | H | H | 4-I | N | T |
| I-21 | H | H | H | H | 4-CF₃ | N | C |
| I-22 | H | H | H | H | 4-CF₃ | N | T |
| I-23 | H | H | H | H | 3-CF₃ | N | C |
| I-24 | H | H | H | H | 3-CF₃ | N | T |
| I-25 | H | H | H | H | 2-CF₃ | N | C |
| I-26 | H | H | H | H | 2-CF₃ | N | T |
| I-27 | H | H | H | H | 4-CH₃ | N | C |
| I-28 | H | H | H | H | 4-CH₃ | N | T |
| I-29 | H | H | H | H | 3-CH₃ | N | C |
| I-30 | H | H | H | H | 3-CH₃ | N | T |
| I-31 | H | H | H | H | 2-CH₃ | N | C |
| I-32 | H | H | H | H | 2-CH₃ | N | T |
| I-33 | H | H | H | H | 4-OCH₃ | N | C |

^{)}“—” (em dash) means unsubstituted (n = 0). The numeric before “-” (en dash) means, when the substituent is on the phenyl ring, the binding position while regarding the position of the binding to the cyclopentane ring through the methylene bridge as 1-position.

TABLE 2

| Compound No | R¹ | R² | R³ | R⁴ | Xn^{)} | A | Isomer type |
|---|---|---|---|---|---|---|---|
| I-34 | H | H | H | H | 4-OCH₃ | N | T |
| I-35 | H | H | H | H | 3-OCH₃ | N | C |
| I-36 | H | H | H | H | 3-OCH₃ | N | T |
| I-37 | H | H | H | H | 2-OCH₃ | N | C |
| I-38 | H | H | H | H | 2-OCH₃ | N | T |
| I-39 | H | H | H | H | 4-OCF₃ | N | C |
| I-40 | H | H | H | H | 4-OCF₃ | N | T |
| I-41 | H | H | H | H | 3-OCF₃ | N | C |
| I-42 | H | H | H | H | 3-OCF₃ | N | T |
| I-43 | H | H | H | H | 2-OCF₃ | N | C |
| I-44 | H | H | H | H | 2-OCF₃ | N | T |
| I-45 | H | H | H | H | 4-OCH₂CF₃ | N | C |
| I-46 | H | H | H | H | 4-OCH₂CF₃ | N | T |
| I-47 | H | H | H | H | 3-OCH₂CF₃ | N | C |
| I-48 | H | H | H | H | 3-OCH₂CF₃ | N | T |
| I-49 | H | H | H | H | 4-Ph | N | C |
| I-50 | H | H | H | H | 4-Ph | N | T |
| I-51 | H | H | H | H | 3-Ph | N | C |
| I-52 | H | H | H | H | 3-Ph | N | T |
| I-53 | H | H | H | H | 4-CN | N | C |
| I-54 | H | H | H | H | 3-CN | N | C |
| I-55 | H | H | H | H | 4-NO₂ | N | C |
| I-56 | H | H | H | H | 3-NO₂ | N | C |
| I-57 | H | H | H | H | 2-F, 4-F | N | C |
| I-58 | H | H | H | H | 2-F, 4-F | N | T |
| I-59 | H | H | H | H | 3-F, 4-F | N | C |
| I-60 | H | H | H | H | 3-F, 4-F | N | T |
| I-61 | H | H | H | H | 2-Cl, 4-Cl | N | C |
| I-62 | H | H | H | H | 2-Cl, 4-Cl | N | T |
| I-63 | H | H | H | H | 3-Cl, 4-Cl | N | C |
| I-64 | H | H | H | H | 3-Cl, 4-Cl | N | T |
| I-65 | H | H | H | H | — | N | C |
| I-66 | H | H | H | H | — | N | T |

TABLE 3

| Compound No | R¹ | R² | R³ | R⁴ | Xn^{)} | A | Isomer type |
|---|---|---|---|---|---|---|---|
| I-101 | H | H | Me | Me | 4-Cl | N | C |
| I-102 | H | H | Me | Me | 4-Cl | N | T |
| I-103 | H | H | Me | Me | 3-Cl | N | C |
| I-104 | H | H | Me | Me | 3-Cl | N | T |
| I-105 | H | H | Me | Me | 2-Cl | N | C |
| I-106 | H | H | Me | Me | 2-Cl | N | T |
| I-107 | H | H | Me | Me | 4-F | N | C |
| I-108 | H | H | Me | Me | 4-F | N | T |
| I-109 | H | H | Me | Me | 3-F | N | C |
| I-110 | H | H | Me | Me | 3-F | N | T |
| I-111 | H | H | Me | Me | 2-F | N | C |
| I-112 | H | H | Me | Me | 2-F | N | T |
| I-113 | H | H | Me | Me | 4-Br | N | C |
| I-114 | H | H | Me | Me | 4-Br | N | T |
| I-115 | H | H | Me | Me | 3-Br | N | C |
| I-116 | H | H | Me | Me | 3-Br | N | T |
| I-117 | H | H | Me | Me | 4-CF₃ | N | C |
| I-118 | H | H | Me | Me | 4-CF₃ | N | T |
| I-119 | H | H | Me | Me | 3-CF₃ | N | C |
| I-120 | H | H | Me | Me | 3-CF₃ | N | T |
| I-121 | H | H | Me | Me | 4-CH₃ | N | C |
| I-122 | H | H | Me | Me | 4-CH₃ | N | T |
| I-123 | H | H | Me | Me | 4-OCF₃ | N | C |
| I-124 | H | H | Me | Me | 4-OCF₃ | N | T |
| I-125 | H | H | Me | Me | 3-OCF₃ | N | C |

TABLE 4

| Compound No | R¹ | R² | R³ | R⁴ | Xn^{)} | A | Isomer type |
|---|---|---|---|---|---|---|---|
| I-126 | H | H | Me | Me | 3-OCF₃ | N | T |
| I-127 | H | H | Me | Me | 4-OCH₂CF₃ | N | C |
| I-128 | H | H | Me | Me | 4-OCH₂CF₃ | N | T |
| I-129 | H | H | Me | Me | 4-Ph | N | C |
| I-130 | H | H | Me | Me | 4-Ph | N | T |
| I-131 | H | H | Me | Me | 2-F, 4-F | N | C |
| I-132 | H | H | Me | Me | 2-F, 4-F | N | T |
| I-133 | H | H | Me | Me | 3-F, 4-F | N | C |
| I-134 | H | H | Me | Me | 3-F, 4-F | N | T |
| I-135 | H | H | Me | Me | 2-Cl, 4-Cl | N | C |
| I-136 | H | H | Me | Me | 2-Cl, 4-Cl | N | T |
| I-137 | H | H | Me | Me | 3-Cl, 4-Cl | N | C |
| I-138 | H | H | Me | Me | 3-Cl, 4-Cl | N | T |
| I-139 | H | H | Me | Me | — | N | C |
| I-140 | H | H | Me | Me | — | N | T |
| I-141 | H | H | H | Me | 4-Cl | N | C |
| I-142 | H | H | H | Me | 4-Cl | N | T |
| I-143 | H | H | Me | H | 4-Cl | N | C |
| I-144 | H | H | Me | H | 4-Cl | N | T |
| I-145 | H | Me | H | H | 4-Cl | N | C |
| I-146 | H | Me | H | H | 4-Cl | N | T |
| I-147 | Me | H | H | H | 4-Cl | N | C |
| I-148 | Me | H | H | H | 4-Cl | N | T |
| I-149 | Me | H | Me | H | 4-Cl | N | C |
| I-150 | Me | H | Me | H | 4-Cl | N | T |

TABLE 5

| Compound No | R¹ | R² | R³ | R⁴ | Xn^{)} | A | Isomer type |
|---|---|---|---|---|---|---|---|
| I-151 | Me | Me | H | H | 4-Cl | N | C |
| I-152 | Me | Me | H | H | 4-Cl | N | T |
| I-153 | Me | Me | H | H | 3-Cl | N | C |
| I-154 | Me | Me | H | H | 3-Cl | N | T |
| I-155 | Me | Me | H | H | 2-Cl | N | C |
| I-156 | Me | Me | H | H | 2-Cl | N | T |
| I-157 | Me | Me | H | H | 4-F | N | C |
| I-158 | Me | Me | H | H | 4-F | N | T |
| I-159 | Me | Me | H | H | 3-F | N | C |
| I-160 | Me | Me | H | H | 3-F | N | T |
| I-161 | Me | Me | H | H | 2-F | N | C |
| I-162 | Me | Me | H | H | 2-F | N | T |
| I-163 | Me | Me | H | H | 4-Br | N | C |
| I-164 | Me | Me | H | H | 4-Br | N | T |
| I-165 | Me | Me | H | H | 3-Br | N | C |
| I-166 | Me | Me | H | H | 3-Br | N | T |
| I-167 | Me | Me | H | H | 4-CF₃ | N | C |
| I-168 | Me | Me | H | H | 4-CF₃ | N | T |
| I-169 | Me | Me | H | H | 3-CF₃ | N | C |
| I-170 | Me | Me | H | H | 3-CF₃ | N | T |
| I-171 | Me | Me | H | H | 4-CH₃ | N | C |
| I-172 | Me | Me | H | H | 4-CH₃ | N | T |
| I-173 | Me | Me | H | H | 4-OCF₃ | N | C |
| I-174 | Me | Me | H | H | 4-OCF₃ | N | T |

TABLE 6

| Compound No | R¹ | R² | R³ | R⁴ | Xn^{)} | A | Isomer type |
|---|---|---|---|---|---|---|---|
| I-175 | Me | Me | H | H | 3-OCF₃ | N | C |
| I-176 | Me | Me | H | H | 3-OCF₃ | N | T |
| I-177 | Me | Me | H | H | 4-OCH₂CF₃ | N | C |
| I-178 | Me | Me | H | H | 4-OCH₂CF₃ | N | T |
| I-179 | Me | Me | H | H | 4-Ph | N | C |
| I-180 | Me | Me | H | H | 4-Ph | N | T |
| I-181 | Me | Me | H | H | 2-F, 4-F | N | C |
| I-182 | Me | Me | H | H | 2-F, 4-F | N | T |
| I-183 | Me | Me | H | H | 3-F, 4-F | N | C |
| I-184 | Me | Me | H | H | 3-F, 4-F | N | T |
| I-185 | Me | Me | H | H | 2-Cl, 4-Cl | N | C |
| I-186 | Me | Me | H | H | 2-Cl, 4-Cl | N | T |
| I-187 | Me | Me | H | H | 3-Cl, 4-Cl | N | C |
| I-188 | Me | Me | H | H | 3-Cl, 4-Cl | N | T |
| I-189 | Me | Me | H | H | — | N | C |
| I-190 | Me | Me | H | H | — | N | T |
| I-191 | Cl | Cl | H | Me | 4-Cl | N | C |
| I-192 | Cl | Cl | H | Me | 4-Cl | N | T |
| I-193 | Cl | Cl | Me | H | 4-Cl | N | C |
| I-194 | Cl | Cl | Me | H | 4-Cl | N | T |
| I-195 | H | Me | Cl | Cl | 4-Cl | N | C |
| I-196 | H | Me | Cl | Cl | 4-Cl | N | T |
| I-197 | Me | H | Cl | Cl | 4-Cl | N | C |
| I-198 | Me | H | Cl | Cl | 4-Cl | N | T |

TABLE 7

| Compound No | R¹ | R² | R³ | R⁴ | Xn^{)} | A | Isomer type |
|---|---|---|---|---|---|---|---|
| I-201 | H | H | Cl | Cl | 4-Cl | N | C |
| I-202 | H | H | Cl | Cl | 4-Cl | N | T |
| I-203 | H | H | Cl | Cl | 3-Cl | N | C |
| I-204 | H | H | Cl | Cl | 3-Cl | N | T |
| I-205 | H | H | Cl | Cl | 2-Cl | N | C |
| I-206 | H | H | Cl | Cl | 2-Cl | N | T |
| I-207 | H | H | Cl | Cl | 4-F | N | C |
| I-208 | H | H | Cl | Cl | 4-F | N | T |
| I-209 | H | H | Cl | Cl | 3-F | N | C |
| I-210 | H | H | Cl | Cl | 3-F | N | T |
| I-211 | H | H | Cl | Cl | 2-F | N | C |
| I-212 | H | H | Cl | Cl | 2-F | N | T |
| I-213 | H | H | Cl | Cl | 4-Br | N | C |
| I-214 | H | H | Cl | Cl | 4-Br | N | T |
| I-215 | H | H | Cl | Cl | 3-Br | N | C |
| I-216 | H | H | Cl | Cl | 3-Br | N | T |
| I-217 | H | H | Cl | Cl | 4-CF₃ | N | C |
| I-218 | H | H | Cl | Cl | 4-CF₃ | N | T |
| I-219 | H | H | Cl | Cl | 3-CF₃ | N | C |
| I-220 | H | H | Cl | Cl | 3-CF₃ | N | T |

TABLE 8

| Compound No | R¹ | R² | R³ | R⁴ | Xn^{)} | A | Isomer type |
|---|---|---|---|---|---|---|---|
| I-221 | H | H | Cl | Cl | 4-CH₃ | N | C |
| I-222 | H | H | Cl | Cl | 4-CH₃ | N | T |
| I-223 | H | H | Cl | Cl | 4-OCF₃ | N | C |
| I-224 | H | H | Cl | Cl | 4-OCF₃ | N | T |
| I-225 | H | H | Cl | Cl | 3-OCF₃ | N | C |
| I-226 | H | H | Cl | Cl | 3-OCF₃ | N | T |
| I-227 | H | H | Cl | Cl | 4-OCH₂CF₃ | N | C |
| I-228 | H | H | Cl | Cl | 4-OCH₂CF₃ | N | T |
| I-229 | H | H | Cl | Cl | 4-Ph | N | C |
| I-230 | H | H | Cl | Cl | 4-Ph | N | T |
| I-231 | H | H | Cl | Cl | 2-F, 4-F | N | C |
| I-232 | H | H | Cl | Cl | 2-F, 4-F | N | T |
| I-233 | H | H | Cl | Cl | 3-F, 4-F | N | C |
| I-234 | H | H | Cl | Cl | 3-F, 4-F | N | T |
| I-235 | H | H | Cl | Cl | 2-Cl, 4-Cl | N | C |
| I-236 | H | H | Cl | Cl | 2-Cl, 4-Cl | N | T |
| I-237 | H | H | Cl | Cl | 3-Cl, 4-Cl | N | C |
| I-238 | H | H | Cl | Cl | 3-Cl, 4-Cl | N | T |
| I-239 | H | H | Cl | Cl | — | N | C |
| I-240 | H | H | Cl | Cl | — | N | T |

TABLE 9

| Compound No | R¹ | R² | R³ | R⁴ | Xn^{)} | A | Isomer type |
|---|---|---|---|---|---|---|---|
| I-241 | Cl | Cl | H | H | 4-Cl | N | C |
| I-242 | Cl | Cl | H | H | 4-Cl | N | T |
| I-243 | Cl | Cl | H | H | 3-Cl | N | C |
| I-244 | Cl | Cl | H | H | 3-Cl | N | T |
| I-245 | Cl | Cl | H | H | 2-Cl | N | C |
| I-246 | Cl | Cl | H | H | 2-Cl | N | T |
| I-247 | Cl | Cl | H | H | 4-F | N | C |
| I-248 | Cl | Cl | H | H | 4-F | N | T |
| I-249 | Cl | Cl | H | H | 3-F | N | C |
| I-250 | Cl | Cl | H | H | 3-F | N | T |
| I-251 | Cl | Cl | H | H | 2-F | N | C |
| I-252 | Cl | Cl | H | H | 2-F | N | T |
| I-253 | Cl | Cl | H | H | 4-Br | N | C |
| I-254 | Cl | Cl | H | H | 4-Br | N | T |
| I-255 | Cl | Cl | H | H | 3-Br | N | C |
| I-256 | Cl | Cl | H | H | 3-Br | N | T |
| I-257 | Cl | Cl | H | H | 4-CF₃ | N | C |
| I-258 | Cl | Cl | H | H | 4-CF₃ | N | T |
| I-259 | Cl | Cl | H | H | 3-CF₃ | N | C |
| I-260 | Cl | Cl | H | H | 3-CF₃ | N | T |

TABLE 10

| Compound No | R¹ | R² | R³ | R⁴ | Xn^{)} | A | Isomer type |
|---|---|---|---|---|---|---|---|
| I-261 | Cl | Cl | H | H | 4-CH₃ | N | C |
| I-262 | Cl | Cl | H | H | 4-CH₃ | N | T |
| I-263 | Cl | Cl | H | H | 4-OCF₃ | N | C |
| I-264 | Cl | Cl | H | H | 4-OCF₃ | N | T |
| I-265 | Cl | Cl | H | H | 3-OCF₃ | N | C |
| I-266 | Cl | Cl | H | H | 3-OCF₃ | N | T |
| I-267 | Cl | Cl | H | H | 4-OCH₂CF₃ | N | C |
| I-268 | Cl | Cl | H | H | 4-OCH₂CF₃ | N | T |
| I-269 | Cl | Cl | H | H | 4-Ph | N | C |
| I-270 | Cl | Cl | H | H | 4-Ph | N | T |
| I-271 | Cl | Cl | H | H | 2-F, 4-F | N | C |
| I-272 | Cl | Cl | H | H | 2-F, 4-F | N | T |
| I-273 | Cl | Cl | H | H | 3-F, 4-F | N | C |
| I-274 | Cl | Cl | H | H | 3-F, 4-F | N | T |
| I-275 | Cl | Cl | H | H | 2-Cl, 4-Cl | N | C |
| I-276 | Cl | Cl | H | H | 2-Cl, 4-Cl | N | T |
| I-277 | Cl | Cl | H | H | 3-Cl, 4-Cl | N | C |
| I-278 | Cl | Cl | H | H | 3-Cl, 4-Cl | N | T |
| I-279 | Cl | Cl | H | H | — | N | C |
| I-280 | Cl | Cl | H | H | — | N | T |

TABLE 11

| Compound No | R¹ | R² | R³ | R⁴ | Xn^{)} | A | Isomer type |
|---|---|---|---|---|---|---|---|
| I-301 | H | H | H | H | 4-Cl | CH | C |
| I-302 | H | H | H | H | 4-Cl | CH | T |
| I-303 | H | H | H | H | 3-Cl | CH | C |
| I-304 | H | H | H | H | 3-Cl | CH | T |
| I-305 | H | H | H | H | 2-Cl | CH | C |
| I-306 | H | H | H | H | 2-Cl | CH | T |
| I-307 | H | H | H | H | 4-F | CH | C |
| I-308 | H | H | H | H | 4-F | CH | T |
| I-309 | H | H | H | H | 3-F | CH | C |
| I-310 | H | H | H | H | 3-F | CH | T |
| I-311 | H | H | H | H | 2-F | CH | C |
| I-312 | H | H | H | H | 2-F | CH | T |
| I-313 | H | H | H | H | 4-Br | CH | C |
| I-314 | H | H | H | H | 4-Br | CH | T |
| I-315 | H | H | H | H | 3-Br | CH | C |
| I-316 | H | H | H | H | 3-Br | CH | T |
| I-317 | H | H | H | H | 2-Br | CH | C |
| I-318 | H | H | H | H | 2-Br | CH | T |
| I-319 | H | H | H | H | 4-I | CH | C |
| I-320 | H | H | H | H | 4-I | CH | T |
| I-321 | H | H | H | H | 4-CF₃ | CH | C |
| I-322 | H | H | H | H | 4-CF₃ | CH | T |
| I-323 | H | H | H | H | 3-CF₃ | CH | C |
| I-324 | H | H | H | H | 3-CF₃ | CH | T |
| I-325 | H | H | H | H | 2-CF₃ | CH | C |
| I-326 | H | H | H | H | 2-CF₃ | CH | T |
| I-327 | H | H | H | H | 4-CH₃ | CH | C |
| I-328 | H | H | H | H | 4-CH₃ | CH | T |
| I-329 | H | H | H | H | 3-CH₃ | CH | C |
| I-330 | H | H | H | H | 3-CH₃ | CH | T |
| I-331 | H | H | H | H | 2-CH₃ | CH | C |
| I-332 | H | H | H | H | 2-CH₃ | CH | T |
| I-333 | H | H | H | H | 4-OCH₃ | CH | C |
| I-334 | H | H | H | H | 4-OCH₃ | CH | T |

TABLE 12

| Compound No | R¹ | R² | R³ | R⁴ | Xn^{)} | A | Isomer type |
|---|---|---|---|---|---|---|---|
| I-335 | H | H | H | H | 3-OCH₃ | CH | C |
| I-336 | H | H | H | H | 3-OCH₃ | CH | T |
| I-337 | H | H | H | H | 2-OCH₃ | CH | C |
| I-338 | H | H | H | H | 2-OCH₃ | CH | T |
| I-339 | H | H | H | H | 4-OCF₃ | CH | C |
| I-340 | H | H | H | H | 4-OCF₃ | CH | T |
| I-341 | H | H | H | H | 3-OCF₃ | CH | C |
| I-342 | H | H | H | H | 3-OCF₃ | CH | T |
| I-343 | H | H | H | H | 4-OCH₂CF₃ | CH | C |
| I-344 | H | H | H | H | 4-OCH₂CF₃ | CH | T |
| I-345 | H | H | H | H | 4-OCH₂CF₃ | CH | C |
| I-346 | H | H | H | H | 4-OCH₂CF₃ | CH | T |
| I-347 | H | H | H | H | 3-OCH₂CF₃ | CH | C |
| I-348 | H | H | H | H | 3-OCH₂CF₃ | CH | T |
| I-349 | H | H | H | H | 4-Ph | CH | C |
| I-350 | H | H | H | H | 4-Ph | CH | T |
| I-351 | H | H | H | H | 3-Ph | CH | C |
| I-352 | H | H | H | H | 3-Ph | CH | T |
| I-353 | H | H | H | H | 4-CN | CH | C |
| I-354 | H | H | H | H | 3-CN | CH | C |
| I-355 | H | H | H | H | 4-NO₂ | CH | C |
| I-356 | H | H | H | H | 3-NO₂ | CH | C |
| I-355 | H | H | H | H | 2-F, 4-F | CH | C |
| I-358 | H | H | H | H | 2-F, 4-F | CH | T |
| I-359 | H | H | H | H | 3-F, 4-F | CH | C |
| I-360 | H | H | H | H | 3-F, 4-F | CH | T |
| I-361 | H | H | H | H | 2-Cl, 4-Cl | CH | C |
| I-362 | H | H | H | H | 2-Cl, 4-Cl | CH | T |
| I-363 | H | H | H | H | 3-Cl, 4-Cl | CH | C |
| I-364 | H | H | H | H | 3-Cl, 4-Cl | CH | T |
| I-365 | H | H | H | H | — | CH | C |
| I-366 | H | H | H | H | — | CH | T |

TABLE 13

| Compound No | R¹ | R² | R³ | R⁴ | Xn^{)} | A | Isomer type |
|---|---|---|---|---|---|---|---|
| I-401 | H | H | Me | Me | 4-Cl | CH | C |
| I-402 | H | H | Me | Me | 4-Cl | CH | T |
| I-403 | H | H | Me | Me | 3-Cl | CH | C |
| I-404 | H | H | Me | Me | 3-Cl | CH | T |
| I-405 | H | H | Me | Me | 2-Cl | CH | C |
| I-406 | H | H | Me | Me | 2-Cl | CH | T |
| I-407 | H | H | Me | Me | 4-F | CH | C |
| I-408 | H | H | Me | Me | 4-F | CH | T |
| I-409 | H | H | Me | Me | 3-F | CH | C |
| I-410 | H | H | Me | Me | 3-F | CH | T |
| I-411 | H | H | Me | Me | 2-F | CH | C |
| I-412 | H | H | Me | Me | 2-F | CH | T |
| I-413 | H | H | Me | Me | 4-Br | CH | C |
| I-414 | H | H | Me | Me | 4-Br | CH | T |
| I-415 | H | H | Me | Me | 3-Br | CH | C |
| I-416 | H | H | Me | Me | 3-Br | CH | T |
| I-417 | H | H | Me | Me | 4-CF₃ | CH | C |
| I-418 | H | H | Me | Me | 4-CF₃ | CH | T |
| I-419 | H | H | Me | Me | 3-CF₃ | CH | C |
| I-420 | H | H | Me | Me | 3-CF₃ | CH | T |
| I-421 | H | H | Me | Me | 4-CH₃ | CH | C |
| I-422 | H | H | Me | Me | 4-CH₃ | CH | T |
| I-423 | H | H | Me | Me | 4-OCF₃ | CH | C |
| I-424 | H | H | Me | Me | 4-OCF₃ | CH | T |
| I-425 | H | H | Me | Me | 3-OCF₃ | CH | C |

TABLE 14

| Compound No | R¹ | R² | R³ | R⁴ | Xn^{)} | A | Isomer type |
|---|---|---|---|---|---|---|---|
| I-426 | H | H | Me | Me | 3-OCF₃ | CH | T |
| I-427 | H | H | Me | Me | 4-OCH₂CF₃ | CH | C |
| I-428 | H | H | Me | Me | 4-OCH₂CF₃ | CH | T |
| I-429 | H | H | Me | Me | 4-Ph | CH | C |
| I-430 | H | H | Me | Me | 4-Ph | CH | T |
| I-431 | H | H | Me | Me | 2-F, 4-F | CH | C |
| I-432 | H | H | Me | Me | 2-F, 4-F | CH | T |
| I-433 | H | H | Me | Me | 3-F, 4-F | CH | C |
| I-434 | H | H | Me | Me | 3-F, 4-F | CH | T |
| I-435 | H | H | Me | Me | 2-Cl, 4-Cl | CH | C |
| I-436 | H | H | Me | Me | 2-Cl, 4-Cl | CH | T |
| I-437 | H | H | Me | Me | 3-Cl, 4-Cl | CH | C |
| I-438 | H | H | Me | Me | 3-Cl, 4-Cl | CH | T |
| I-439 | H | H | Me | Me | — | CH | C |
| I-440 | H | H | Me | Me | — | CH | T |
| I-441 | H | H | H | Me | 4-Cl | CH | C |
| I-442 | H | H | H | Me | 4-Cl | CH | T |
| I-443 | H | H | Me | H | 4-Cl | CH | C |
| I-444 | H | H | Me | H | 4-Cl | CH | T |
| I-445 | H | Me | H | H | 4-Cl | CH | C |
| I-446 | H | Me | H | H | 4-Cl | CH | T |
| I-447 | Me | H | H | H | 4-Cl | CH | C |
| I-448 | Me | H | H | H | 4-Cl | CH | T |
| I-449 | Me | H | Me | H | 4-Cl | CH | C |
| I-450 | Me | H | Me | H | 4-Cl | CH | T |

TABLE 15

| Compound No | R¹ | R² | R³ | R⁴ | Xn^{)} | A | Isomer type |
|---|---|---|---|---|---|---|---|
| I-451 | Me | Me | H | H | 4-Cl | CH | C |
| I-452 | Me | Me | H | H | 4-Cl | CH | T |
| I-453 | Me | Me | H | H | 3-Cl | CH | C |
| I-454 | Me | Me | H | H | 3-Cl | CH | T |
| I-455 | Me | Me | H | H | 2-Cl | CH | C |
| I-456 | Me | Me | H | H | 2-Cl | CH | T |
| I-457 | Me | Me | H | H | 4-F | CH | C |
| I-458 | Me | Me | H | H | 4-F | CH | T |
| I-459 | Me | Me | H | H | 3-F | CH | C |
| I-460 | Me | Me | H | H | 3-F | CH | T |
| I-461 | Me | Me | H | H | 2-F | CH | C |
| I-462 | Me | Me | H | H | 2-F | CH | T |
| I-463 | Me | Me | H | H | 4-Br | CH | C |
| I-464 | Me | Me | H | H | 4-Br | CH | T |
| I-465 | Me | Me | H | H | 3-Br | CH | C |
| I-466 | Me | Me | H | H | 3-Br | CH | T |
| I-467 | Me | Me | H | H | 4-CF₃ | CH | C |
| I-468 | Me | Me | H | H | 4-CF₃ | CH | T |
| I-469 | Me | Me | H | H | 3-CF₃ | CH | C |
| I-470 | Me | Me | H | H | 3-CF₃ | CH | T |
| I-471 | Me | Me | H | H | 4-CH₃ | CH | C |
| I-472 | Me | Me | H | H | 4-CH₃ | CH | T |
| I-473 | Me | Me | H | H | 4-OCF₃ | CH | C |
| I-474 | Me | Me | H | H | 4-OCF₃ | CH | T |

TABLE 16

| Compound No | R¹ | R² | R³ | R⁴ | Xn^{)} | A | Isomer type |
|---|---|---|---|---|---|---|---|
| I-475 | Me | Me | H | H | 3-OCF₃ | CH | C |
| I-476 | Me | Me | H | H | 3-OCF₃ | CH | T |
| I-477 | Me | Me | H | H | 4-OCH₂CF₃ | CH | C |
| I-478 | Me | Me | H | H | 4-OCH₂CF₃ | CH | T |
| I-479 | Me | Me | H | H | 4-Ph | CH | C |
| I-480 | Me | Me | H | H | 4-Ph | CH | T |
| I-481 | Me | Me | H | H | 2-F, 4-F | CH | C |
| I-482 | Me | Me | H | H | 2-F, 4-F | CH | T |
| I-483 | Me | Me | H | H | 3-F, 4-F | CH | C |
| I-484 | Me | Me | H | H | 3-F, 4-F | CH | T |
| I-485 | Me | Me | H | H | 2-Cl, 4-Cl | CH | C |
| I-486 | Me | Me | H | H | 2-Cl, 4-Cl | CH | T |
| I-487 | Me | Me | H | H | 3-Cl, 4-Cl | CH | C |
| I-488 | Me | Me | H | H | 3-Cl, 4-Cl | CH | T |
| I-489 | Me | Me | H | H | — | CH | C |
| I-490 | Me | Me | H | H | — | CH | T |
| I-491 | Cl | Cl | H | Me | 4-Cl | CH | C |
| I-492 | Cl | Cl | H | Me | 4-Cl | CH | T |
| I-493 | Cl | Cl | Me | H | 4-Cl | CH | C |
| I-494 | Cl | Cl | Me | H | 4-Cl | CH | T |
| I-495 | H | Me | Cl | Cl | 4-Cl | CH | C |
| I-496 | H | Me | Cl | Cl | 4-Cl | CH | T |
| I-497 | Me | H | Cl | Cl | 4-Cl | CH | C |
| I-498 | Me | H | Cl | Cl | 4-Cl | CH | T |

TABLE 17

| Compound No | R¹ | R² | R³ | R⁴ | Xn^{)} | A | Isomer type |
|---|---|---|---|---|---|---|---|
| I-501 | H | H | Cl | Cl | 4-Cl | CH | C |
| I-502 | H | H | Cl | Cl | 4-Cl | CH | T |
| I-503 | H | H | Cl | Cl | 3-Cl | CH | C |
| I-504 | H | H | Cl | Cl | 3-Cl | CH | T |
| I-505 | H | H | Cl | Cl | 2-Cl | CH | C |
| I-506 | H | H | Cl | Cl | 2-Cl | CH | T |
| I-507 | H | H | Cl | Cl | 4-F | CH | C |
| I-508 | H | H | Cl | Cl | 4-F | CH | T |
| I-509 | H | H | Cl | Cl | 3-F | CH | C |
| I-510 | H | H | Cl | Cl | 3-F | CH | T |
| I-511 | H | H | Cl | Cl | 2-F | CH | C |
| I-512 | H | H | Cl | Cl | 2-F | CH | T |
| I-513 | H | H | Cl | Cl | 4-Br | CH | C |
| I-514 | H | H | Cl | Cl | 4-Br | CH | T |
| I-515 | H | H | Cl | Cl | 3-Br | CH | C |
| I-516 | H | H | Cl | Cl | 3-Br | CH | T |
| I-517 | H | H | Cl | Cl | 4-CF₃ | CH | C |
| I-518 | H | H | Cl | Cl | 4-CF₃ | CH | T |
| I-519 | H | H | Cl | Cl | 3-CF₃ | CH | C |
| I-520 | H | H | Cl | Cl | 3-CF₃ | CH | T |

TABLE 18

| Compound No | R¹ | R² | R³ | R⁴ | Xn^{)} | A | Isomer type |
|---|---|---|---|---|---|---|---|
| I-521 | H | H | Cl | Cl | 4-CH₃ | CH | C |
| I-522 | H | H | Cl | Cl | 4-CH₃ | CH | T |
| I-523 | H | H | Cl | Cl | 4-OCF₃ | CH | C |
| I-524 | H | H | Cl | Cl | 4-OCF₃ | CH | T |
| I-525 | H | H | Cl | Cl | 3-OCF₃ | CH | C |
| I-526 | H | H | Cl | Cl | 3-OCF₃ | CH | T |
| I-527 | H | H | Cl | Cl | 4-OCH₂CF₃ | CH | C |
| I-528 | H | H | Cl | Cl | 4-OCH₂CF₃ | CH | T |
| I-529 | H | H | Cl | Cl | 4-Ph | CH | C |
| I-530 | H | H | Cl | Cl | 4-Ph | CH | T |
| I-531 | H | H | Cl | Cl | 2-F, 4-F | CH | C |
| I-532 | H | H | Cl | Cl | 2-F, 4-F | CH | T |
| I-533 | H | H | Cl | Cl | 3-F, 4-F | CH | C |
| I-534 | H | H | Cl | Cl | 3-F, 4-F | CH | T |
| I-535 | H | H | Cl | Cl | 2-Cl, 4-Cl | CH | C |
| I-536 | H | H | Cl | Cl | 2-Cl, 4-Cl | CH | T |
| I-537 | H | H | Cl | Cl | 3-Cl, 4-Cl | CH | C |
| I-538 | H | H | Cl | Cl | 3-Cl, 4-Cl | CH | T |
| I-539 | H | H | Cl | Cl | — | CH | C |
| I-540 | H | H | Cl | Cl | — | CH | T |

TABLE 19

| Compound No | R¹ | R² | R³ | R⁴ | Xn^{)} | A | Isomer type |
|---|---|---|---|---|---|---|---|
| I-541 | Cl | Cl | H | H | 4-Cl | CH | C |
| I-542 | Cl | Cl | H | H | 4-Cl | CH | T |
| I-543 | Cl | Cl | H | H | 3-Cl | CH | C |
| I-544 | Cl | Cl | H | H | 3-Cl | CH | T |
| I-545 | Cl | Cl | H | H | 2-Cl | CH | C |
| I-546 | Cl | Cl | H | H | 2-Cl | CH | T |
| I-547 | Cl | Cl | H | H | 4-F | CH | C |
| I-548 | Cl | Cl | H | H | 4-F | CH | T |
| I-549 | Cl | Cl | H | H | 3-F | CH | C |
| I-550 | Cl | Cl | H | H | 3-F | CH | T |
| I-551 | Cl | Cl | H | H | 2-F | CH | C |
| I-552 | Cl | Cl | H | H | 2-F | CH | T |
| I-553 | Cl | Cl | H | H | 4-Br | CH | C |
| I-554 | Cl | Cl | H | H | 4-Br | CH | T |
| I-555 | Cl | Cl | H | H | 3-Br | CH | C |
| I-556 | Cl | Cl | H | H | 3-Br | CH | T |
| I-557 | Cl | Cl | H | H | 4-CF₃ | CH | C |
| I-558 | Cl | Cl | H | H | 4-CF₃ | CH | T |
| I-559 | Cl | Cl | H | H | 3-CF₃ | CH | C |
| I-560 | Cl | Cl | H | H | 3-CF₃ | CH | T |

TABLE 20

| Compound No | R¹ | R² | R³ | R⁴ | Xn^{)} | A | Isomer type |
|---|---|---|---|---|---|---|---|
| I-561 | Cl | Cl | H | H | 4-CH₃ | CH | C |
| I-562 | Cl | Cl | H | H | 4-CH₃ | CH | T |
| I-563 | Cl | Cl | H | H | 4-OCF₃ | CH | C |
| I-564 | Cl | Cl | H | H | 4-OCF₃ | CH | T |
| I-565 | Cl | Cl | H | H | 3-OCF₃ | CH | C |
| I-566 | Cl | Cl | H | H | 3-OCF₃ | CH | T |
| I-567 | Cl | Cl | H | H | 4-OCH₂CF₃ | CH | C |
| I-568 | Cl | Cl | H | H | 4-OCH₂CF₃ | CH | T |
| I-569 | Cl | Cl | H | H | 4-Ph | CH | C |
| I-570 | Cl | Cl | H | H | 4-Ph | CH | T |
| I-571 | Cl | Cl | H | H | 2-F, 4-F | CH | C |
| I-572 | Cl | Cl | H | H | 2-F, 4-F | CH | T |
| I-573 | Cl | Cl | H | H | 3-F, 4-F | CH | C |
| I-574 | Cl | Cl | H | H | 3-F, 4-F | CH | T |
| I-575 | Cl | Cl | H | H | 2-Cl, 4-Cl | CH | C |
| I-576 | Cl | Cl | H | H | 2-Cl, 4-Cl | CH | T |
| I-577 | Cl | Cl | H | H | 3-Cl, 4-Cl | CH | C |
| I-578 | Cl | Cl | H | H | 3-Cl, 4-Cl | CH | T |
| I-579 | Cl | Cl | H | H | — | CH | C |
| I-580 | Cl | Cl | H | H | — | CH | T |

### B) Method for producing 5-benzyl-4-azolylmethyl-4-spiro[2.4]heptanol derivative

While in the description shown below in relation to the production the solvent employed is not limited particularly, those which may be exemplified include halogenated hydrocarbons such as dichloromethane, chloroform, dichloroethane and the like, aromatic hydrocarbons such as benzene, toluene, xylene and the like, aliphatic hydrocarbons such as petroleum ether, hexane, methylcyclohexane and the like, amides such as N,N-dimethylformamide, N,N-dimethylacetamide, N-methyl-2-pyrrolidinone and the like, ethers such as diethyl ether, tetrahydrofuran, dioxane and the like, alcohols such as methanol, ethanol and the like.

Those which may also be exemplified are water, carbon disulfide, acetonitrile, ethyl acetate, pyridine, dimethyl sulfoxide and the like. Two or more of these solvents may be employed in combination.

One which may also be exemplified is a solvent composition consisting of solvents which do not form a homogenous layer with each other. For example, to a reaction mixture, a quaternary ammonium salt such as tetrabutylammonium salt and a phase transfer catalyst such as a crown ether and analogues are added to effect the reaction thereof. In such a case, the solvents employed are not limited, while the oily phase may consists of benzene, chloroform, dichloromethane, hexane, toluene and the like.

In the description shown below in relation to the production, the reaction may be performed in the presence of a base or an acid in addition to the solvents described above.

In such a case, the base employed is not limited particularly, and may for example be a carbonate of an alkaline metal such as sodium carbonate, sodium hydrogen carbonate, potassium carbonate, potassium hydrogen carbonate and the like; a carbonate of an alkaline earth metal such as calcium carbonate, barium carbonate and the like; a hydroxide of an alkaline metal such as sodium hydroxide, potassium hydroxide and the like; an alkoxide of an alkaline metal such as sodium methoxide, sodium ethoxide, sodium t-butoxide, potassium t-butoxide and the like; an alkaline metal hydride such as sodium hydride, potassium hydride, lithium hydride and the like; an organometal compound of an alkaline metal such as n-butyl lithium and the like; an alkaline metal such as sodium, potassium, lithium and the like; an alkaline metal amide such as lithium diisopropyl amide and the like; and an organic amine such as triethylamine, pyridine, 4-dimethylaminopyridine, N,N-dimethylaniline, 1,8-diazabicyclo-7-[5.4.0]undecene and the like.

Also, the acid employed is not limited particularly, it may for example be an inorganic acid such as hydrochloric acid, hydrobromic acid, hydroiodic acid, sulfuric acid and the like, an organic acid such as formic acid, acetic acid, butyric acid, p-toluenesulfonic acid and the like, a Lewis acid such as lithium chloride, lithium bromide, rhodium chloride and the like.

A method for producing a 5-benzyl-4-azolylmethyl-4-spiro[2.4]heptanol derivative represented by Formula (I) described above is now explained below. Scheme (1) is a scheme illustrating a method for producing a 5-benzyl-4-azolylmethyl-4-spiro[2.4]heptanol derivative according to the invention.

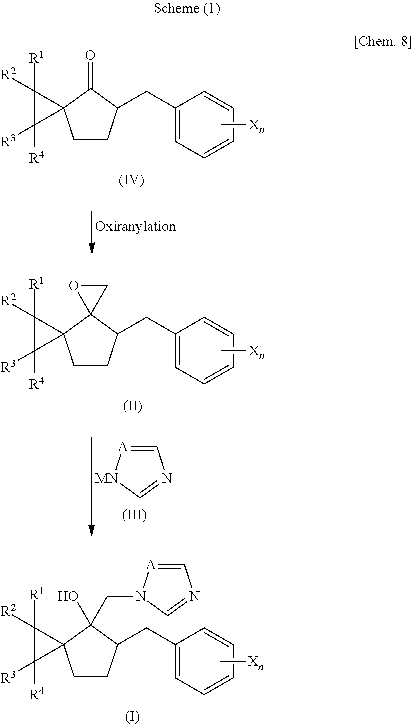

A 5-benzyl-4-azolylmethyl-4-spiro[2.4]heptanol derivative represented by Formula (I) described above is characterized in that an oxirane derivative represented by Formula (II) described above, which is obtained by oxiranylating a carbonyl compound represented by Formula (IV) described above, is reacted with a 1,2,4-triazole or imidazole compound represented by Formula (III) described above, whereby forming a carbon-nitrogen bond between the carbon atom in the oxirane ring of the oxirane derivative described above and the nitrogen atom in the 1,2,4-triazole or imidazole compound (see Scheme (1)).

A method for obtaining an oxirane derivative represented by Formula (II) by oxiranylating a carbonyl compound represented by Formula (IV) is now explained below.

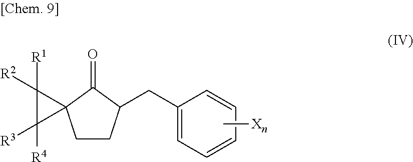

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group.

A preferred first synthesis method of an oxirane derivative represented by Formula (II) employed in the invention may for example be a method in which a carbonyl compound represented by Formula (IV) is reacted with a sulfur ylide such as a sulfonium methylide including dimethyl sulfonium methylide or a sulfoxonium methylide including dimethyl sulfoxonium methylide in a solvent (see Scheme (2)).

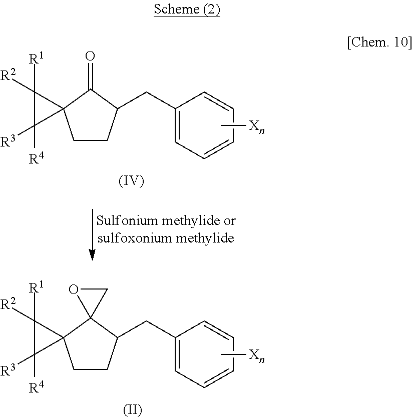

The sulfonium methylides or sulfoxonium methylides used here can be produced by reacting a sulfonium salt (for example, trimethyl sulfonium iodide or trimethyl sulfonium bromide and the like) or a sulfoxonium salt (for example, trimethyl sulfoxonium iodide or trimethyl sulfoxonium bromide and the like) with a base in a solvent.

The amount of the sulfonium methylide or sulfoxonium methylide employed here is 0.5 to 5 moles, preferably 0.8 to 2 moles per mole of the carbonyl compound represented by Formula (IV) described above.

While the solvent employed is not limited particularly, it may for example be dimethyl sulfoxide, an amide such as N-methylpyrrolidone, N,N-dimethylformamide, tetrahydrofuran, dioxane and other ethers, as well as a solvent mixture thereof.

While the base employed for producing the sulfonium methylide or sulfoxonium methylide is not limited particularly, those employed preferably include metal hydrides such as sodium hydride, alkaline metal alkoxides such as sodium methoxide, sodium ethoxide, sodium t-butoxide, potassium t-butoxide and the like.

The reaction temperature of the preferred first synthetic method of the oxirane derivative represented by Formula (II) described above may appropriately be selected depending on the types of the solvent, the carbonyl compound represented by Formula (IV) described above, the sulfonium salt or sulfoxonium salt, bases employed, and is preferably −100 degrees C. (Celsius) to 200 degrees C., more preferably −50 degrees C. to 150 degrees C. The reaction time may appropriately be selected depending on the types of the solvent, the carbonyl compound represented by Formula (IV) described above, the sulfonium salt or sulfoxonium salt, bases employed, and is preferably 0.1 hour to several days, more preferably 0.5 hours to 2 days.

As a preferred second synthesis method of the oxirane derivative represented by Formula (II) employed in the invention, a method in which the carbonyl compound represented by Formula (IV) described above is reacted with samarium diiodide and diiodomethane in a solvent and then treated with a base may be exemplified. The base employed is not limited particularly, and may for example be sodium hydroxide (see Scheme (3)).

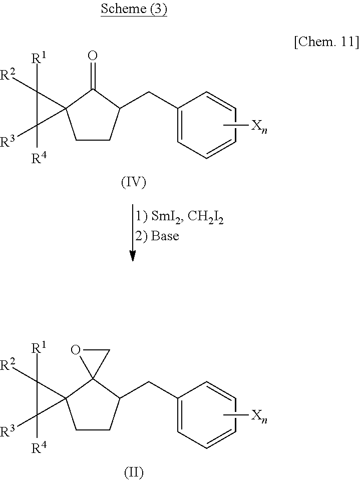

The amount of samarium diiodide employed here is preferably 0.5 to 10 moles, more preferably 1 to 6 moles per mole of the carbonyl compound represented by Formula (IV) described above. The amount of diiodomethane employed here is preferably 0.5 to 10 moles, more preferably 0.8 to 5 moles per mole of the carbonyl compound represented by Formula (IV) described above.

Samarium diiodide employed here can be produced by reacting an elemental samarium with 1,2-diiodoethane or diiodomethane in an anhydrous solvent.

While the amount of samarium diiodide per mole of the carbonyl compound represented by Formula (IV) described above is not limited particularly, it is preferably 0.5 to 10 moles, more preferably 0.8 to 6 moles. The preferred solvent employed in this reaction is not limited particularly, and it may for example be an ether such as tetrahydrofuran and the like.

The reaction temperature of the preferred second synthetic method of the oxirane derivative represented by Formula (II) described above may appropriately be selected depending on the types of the solvent, the carbonyl compound represented by Formula (IV) described above, the base employed, and is preferably −100 degrees C. to 150 degrees C., more preferably −50 degrees C. to 100 degrees C. The reaction time may appropriately be selected depending on the types of the solvent, the carbonyl compound represented by Formula (IV) described above, the base employed, and is preferably 0.1 hour to several days, more preferably 0.5 hours to 2 days.

A method for obtaining an 5-benzyl-4-azolylmethyl-4-spiro[2.4]heptanol derivative by reacting an oxirane derivative represented by Formula (II) described above with a 1,2,4-triazole or imidazole compound represented by Formula (III) described above is now explained below.

It is preferred to mix an oxirane derivative represented by Formula (II) described above with a 1,2,4-triazole or imidazole compound represented by Formula (III) described above in a solvent to form a carbon-nitrogen bond between the carbon atom in the oxirane ring of the oxirane derivative and the nitrogen atom in the 1,2,4-triazole or imidazole compound (see Scheme (4)).

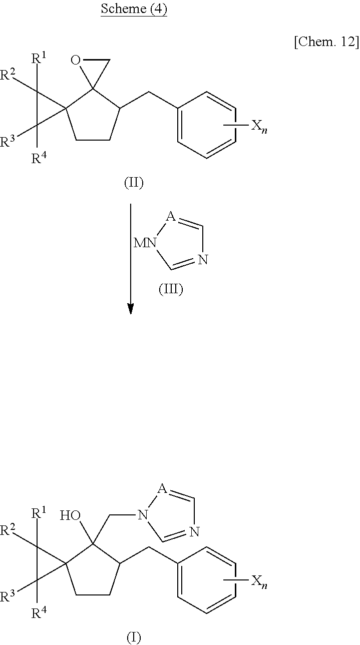

While the solvent employed here is not limited particularly, it may for example be an amide such as N-methylpyrrolidone or N,N-dimethylformamide.

The amount of the compound represented by Formula (III) per mole of the oxirane derivative represented by Formula (II) is usually 0.5 to 10 moles, preferably 0.8 to 5 moles. It is possible to add a base if necessary, and in such a case the amount of the base per the compound represented by Formula (III) is usually greater than 0 up to 5 moles, preferably 0.5 to 2 moles.

The reaction temperature may appropriately be selected depending on the solvent and the base employed, and is preferably 0 degrees C. to 250 degrees C., more preferably 10 degrees C. to 200 degrees C. The reaction time may appropriately be selected depending on the solvent and the base employed, and is preferably 0.1 hour to several days, more preferably 0.5 hours to 2 days.

While there is a method in which an oxirane derivative represented by Formula (II) is produced and then reacted stepwise with a compound represented by Formula (III), the yield may be reduced due for example to generation of byproducts such as an oxetane derivative when the oxiranylating reaction is conducted alone in the method described above as a preferred first synthesis method of an oxirane derivative represented by Formula (II) in which a carbonyl compound represented by Formula (IV) is reacted with a sulfur ylide such as a sulfonium methylide including dimethyl sulfonium methylide or a sulfoxonium methylide including dimethyl sulfoxonium methylide in a solvent. In such a case, a method in which the azolation is conducted while producing the oxirane derivative represented by Formula (II) is preferred (see Scheme (5)).

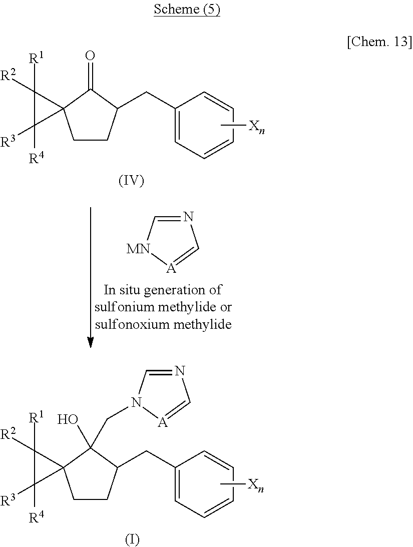

In such a case, a carbonyl compound represented by Formula (IV) described above and an azole compound represented by Formula (III) are dissolved in an amide bond-carrying polar solvent or dimethyl sulfoxide, or a solvent mixture of such a polar solvent with a selected alcohol, to which a trimethyl sulfonium salt or a trimethyl sulfoxonium salt and a base are added intermittently, whereby effecting an in situ generation of a sulfonium methylide including dimethyl sulfonium methylide or a sulfoxonium methylide including dimethyl sulfoxonium methylide whereby accomplishing the azolation while generating the oxirane derivative represented by Formula (II).

The solvent employed is not limited particularly, and one employed preferably may for example be an amide bond-carrying polar solvent such as N-methylpyrrolidone or N,N-dimethylformamide, or dimethyl sulfoxide, or a solvent mixture of such a polar solvent with a selected alcohol such as t-butanol.

The base employed for producing a sulfonium methylide and a sulfoxonium methylide is not limited particularly, and one employed preferably may for example be a metal hydride such as sodium hydride, and an alkoxide of an alkaline metal such as sodium methoxide, sodium ethoxide, sodium t-butoxide, potassium t-butoxide and the like. An alkaline metal salt of 1,2,4-triazole and imidazole may also be employed.

The reaction temperature of the synthesis method in which a carbonyl compound represented by Formula (IV) described above and an azole compound represented by Formula (III) are dissolved in an amide bond-carrying polar solvent or dimethyl sulfoxide, or a solvent mixture of such a polar solvent with a selected alcohol, to which a trimethyl sulfonium halide or a trimethyl sulfoxonium halide and a base are added intermittently whereby accomplishing the azolation while generating the oxirane derivative represented by Formula (II) may appropriately be selected depending on the types of the solvent, the carbonyl compound represented by Formula (IV) described above, the sulfonium salt or sulfoxonium salt, bases employed, and is preferably −100 degrees C. to 250 degrees C., more preferably −50 degrees C. to 200 degrees C. The reaction time may appropriately be selected depending on the types of the solvent, the carbonyl compound represented by Formula (IV) described above, the sulfonium salt or sulfoxonium salt, bases employed, and is preferably 0.1 hour to several days, more preferably 0.5 hours to 2 days. The number of times the trimethyl sulfonium halide or the trimethyl sulfoxonium halide and the base are added intermittently is not limited particularly as long as a certain purpose is achieved, and may usually be 2 to 20 times, preferably 3 to 15 times.

In such a case, the total amount of the sulfonium salt or sulfoxonium salt is preferably 0.5 to 5 moles, more preferably 0.8 to 2 moles per the carbonyl compound represented by Formula (IV) described above. The amount of the compound represented by Formula (III) per mole of the carbonyl compound represented by Formula (IV) is usually 0.5 to 10 moles, preferably 0.8 to 5 moles. It is further preferred to use a compound represented by Formula (III) in which the M is an alkaline metal.

A method for producing a certain azolylmethylcycloalkanol derivative in which the azolation is conducted while generating an oxirane derivative is described in JP-A 1-301664.

As a preferred first synthesis method of a carbonyl compound represented by Formula (IV) described above, a method in which a 2-(2-haloethyl)cyclopentanone compound represented by Formula (V) is subjected to an intramolecular nucleophilic substitution reaction in a solvent in the presence of a base may be exemplified (see Scheme (6)).

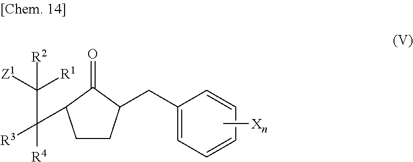

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and Z¹ denotes a halogen atom.

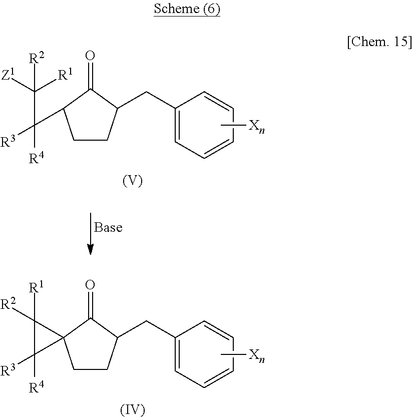

The base employed here is not limited particularly, and may for example be an alkaline metal hydride such as sodium hydride and the like, an alkaline metal carbonate such as sodium carbonate, potassium carbonate and the like, and an alkaline metal hydroxide such as sodium hydroxide, potassium hydroxide and the like.

The reaction temperature of the preferred first synthetic method of the carbonyl compound represented by Formula (IV) described above may appropriately be selected depending on the solvent and the base employed, and is preferably −50 degrees C. to 250 degrees C., more preferably 0 degrees C. to 150 degrees C. The reaction time may appropriately be selected depending on the solvent and the base employed, and is preferably 0.1 hour to several days, more preferably 0.5 hours to 2 days.

As a preferred first synthesis method of a 2-(2-haloethyl)cyclopentanone compound represented by Formula (V) described above, a method comprising a step in which a ketoester compound represented by Formula (VII) and a dihalogenoalkane compound represented by Formula (VIII) are reacted to obtain a haloalkylated ketoester compound represented by Formula (VI) (hereinafter referred to as Step A) and a step in which an alkoxycarbonyl group is hydrolyzed and decarboxylated (hereinafter referred to as Step B) may be exemplified (see Scheme (7)).

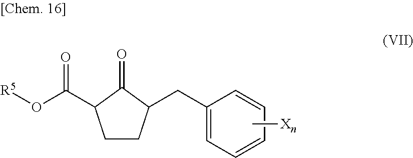

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; and R⁵ denotes a C₁-C₄ alkyl group.

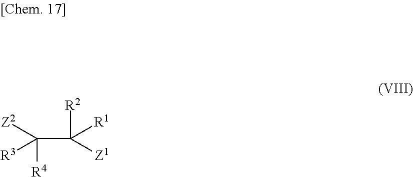

wherein R¹, R², R³, R⁴ each independently denotes a hydrogen atom, a halogen atom or a C₁-C₅ alkyl group; and Z¹, Z² each independently denotes a halogen atom.

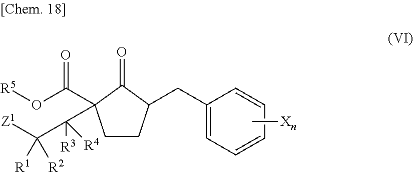

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; R⁵ denotes a C1-C4 alkyl group; and Z¹ denotes a halogen atom.

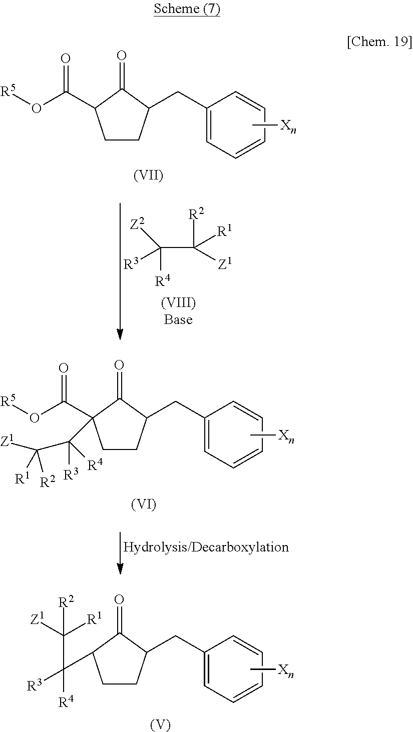

Step A is conducted by reacting the ketoester compound represented by Formula (VII) and the dihalogenoalkane compound represented by Formula (VIII) in a solvent in the presence of a base.

The base employed here is not limited particularly, and may for example be an alkaline metal hydride such as sodium hydride and the like, an alkaline metal carbonate such as sodium carbonate, potassium carbonate and the like. The amount of the base employed is preferably 0.5 to 5 moles, more preferably 0.8 to 2 moles per the ketoester compound represented by Formula (VII).

The amount of the dihalogenoalkane compound represented by Formula (VIII) described above is preferably 0.5 to 10 moles, more preferably 0.8 to 5 moles per mole of the ketoester compound represented by Formula (VII).

In the ketoester compound represented by Formula (VII) described above, R⁵ is preferably a methyl group or an ethyl group. This ketoester compound can be synthesized by a known method such as one described in JP-A 5-78282 (corresponding to EP0537909 etc.). Compounds (VI) in which X=4-Cl, n=1, R¹=H, R²=H, R³=H, R⁴=H, Z¹=F and Compounds (V) in which X=4-Cl, n=1, R¹=H, R²=H, R³=H, R⁴=H, Z¹=F are described in JP-A 2-72176.

The reaction temperature of Step A may appropriately be selected depending on the solvent, the ketoester compound represented by Formula (VII) described above, the dihalogenoalkane compound represented by Formula (VIII) described above, and the base employed, and is preferably 0 degrees C. to 250 degrees C., more preferably room temperature to 150 degrees C. The reaction time may appropriately be selected depending on the solvent, the ketoester compound represented by Formula (VII) described above, the dihalogenoalkane compound represented by Formula (VIII) described above, and the base employed, and is preferably 0.1 hour to several days, more preferably 0.5 hours to 24 hours.

Step B is conducted by subjecting an alkoxycarbonyl group of the haloalkylated ketoester compound represented by Formula (VI) described above to a hydrolysis/decarboxylation in a solvent under an acidic condition.

The acid employed here is not limited particularly, and is preferably an inorganic acid such as hydrochloric acid, hydrobromic acid, sulfuric acid and the like. The solvent employed is not limited particularly, and may be water with or without an organic acid such as acetic acid.

The reaction temperature of Step B may appropriately be selected depending on the solvent, the haloalkylated ketoester compound represented by Formula (VI) described above, and the acid catalyst employed, and is preferably 0 degrees C. to reflux temperature, more preferably room temperature to reflux temperature. The reaction time may appropriately be selected depending on the solvent, the haloalkylated ketoester compound represented by Formula (VI) described above, and the acid catalyst employed, and is preferably 0.1 hour to several days, more preferably 0.5 hours to 24 hours.

As a preferred second synthesis method of the carbonyl compound represented by Formula (IV) described above, a method comprising a step in which a cyclopentanone compound represented by Formula (X) and a compound represented by Formula (XI) are subjected to an aldol condensation reaction to obtain an alkylidene compound represented by Formula (IX) (hereinafter referred to as Step C) followed by a step in which a carbon-carbon double bond is subjected to a cyclopropanation (hereinafter referred to as Step D) may be exemplified (see Scheme (8)).

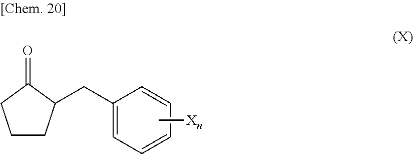

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different.

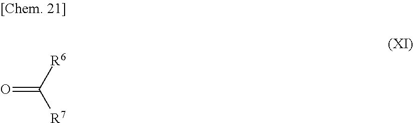

wherein R⁶, R⁷ each denotes a hydrogen atom or a C₁-C₅ alkyl group.

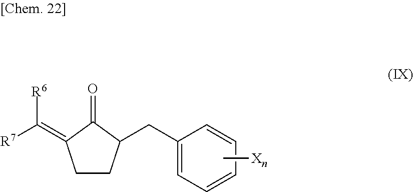

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different: and R⁶, R⁷ each denotes a hydrogen atom or a C₁-C₅ alkyl group.

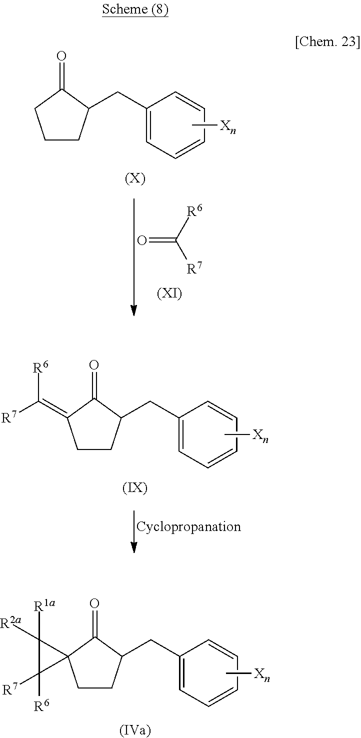

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R⁶, R⁷ each denotes a hydrogen atom or a C₁-C₅ alkyl group; and R^{1a}, R^{2a} each denotes a hydrogen atom, a halogen atom or C₁-C₅ alkyl group.

Step C is conducted by subjecting the cyclopentanone compound represented by Formula (X) described above and the compound represented by Formula (XI) described above to an aldol condensation reaction in a solvent in the presence of a base or an acid.

The base or acid employed here is not limited particularly, and may preferably be an alkaline metal hydroxide such as sodium hydroxide, potassium hydroxide and the like. The amount of the base or acid employed is preferably 0.01 to 5 moles, more preferably 0.1 to 2 moles per mole of the cyclopentanone compound represented by Formula (X) described above.

The amount of the compound represented by Formula (XI) described above is preferably 0.5 to 10 moles, more preferably 0.8 to 5 moles per mole of the cyclopentanone compound represented by Formula (X) described above.

The cyclopentanone compound represented by Formula (X) described above can be synthesized by a method described in references.

The reaction temperature of Step C may appropriately be selected depending on the solvent, the cyclopentanone compound represented by Formula (X) described above, the compound represented by Formula (XIII) described above, and the base or acid employed, and is preferably 0 degrees C. to 250 degrees C., more preferably room temperature to 150 degrees C. The reaction time may appropriately be selected depending on the solvent, the cyclopentanone compound represented by Formula (X) described above, the compound represented by Formula (XIII) described above, and the base or acid employed, and is preferably 0.1 hour to several days, more preferably 0.5 hours to 24 hours.

In Step D, the cyclopropanation of the carbon-carbon double bond of the alkylidene compound represented by Formula (IX) is conducted for example by (a) reaction with a sulfoxonium ylide such as dimethyl sulfoxonium methylide, (b) reaction of a trihalomethane for example with chloroform and a base such as aqueous solution of sodium hydroxide, or addition reaction of a halocarbene generated by trihaloacetate pyrolysis and the like, or (c) addition reaction of a hydrocarbon-based carbene employing diiodomethane and zinc-copper, diiodomethane and diethylzinc and the like.

When using (a) reaction with a sulfoxonium ylide, for example, the amount of the sulfoxonium ylide employed may appropriately be selected depending on the types of the alkylidene compound represented by Formula (IX) described above, and is preferably 0.05 to 5 moles, more preferably 0.8 to 2 moles per mole of the alkylidene compound represented by Formula (X) described above. When the resultant compound (IVa) undergoes here a reaction with the sulfoxonium ylide under the same condition, an approximately equivalent amount is preferred for the purpose of obtaining the resultant compound (IVa) at a high yield.

The sulfoxonium ylide described above can be produced for example by reaction of a sulfoxonium salt such as trimethylsulfoxonium iodide or trimethylsulfoxonium bromide and a base.

The base employed here is not limited particularly, and may for example be an alkaline metal hydride such as sodium hydride and the like, and an alkaline metal alkoxide such as sodium methoxide, sodium ethoxide, potassium t-butoxide and the like.

The reaction temperature of Step D may appropriately be selected depending on the types of the solvent, the alkylidene compound represented by Formula (IX) described above employed, and is preferably −100 degrees C. to 150 degrees C., more preferably −20 degrees C. to 100 degrees C. The reaction time may appropriately be selected depending on the types of the solvent, the alkylidene compound represented by Formula (IX) described above employed, and is preferably 0.1 hour to several days, more preferably 0.5 hours to 2 days.

In a preferred third synthesis method of the carbonyl compound represented by Formula (IV) described above, a method in which a spiro[2.]4]heptan-4-one compound represented by Formula (XV) is reacted with a compound represented by Formula (XVI) in the presence of a base to obtain a ketoester compound represented by Formula (XIV) (hereinafter referred to as Step E), and then a carbon-carbon bond is formed between the carbon atom to which an alkoxycarbonyl group of Compound (XIV) is bound and the carbon atom to which a halogen atom of a benzyl halide compound represented by Formula (XIII) to give a benzyl ketoester compound represented by Formula (XII) (hereinafter referred to as Step F), and then a hydrolysis/decarboxylation is conducted (hereinafter referred to as Step G) may be conducted (see Scheme (9)).

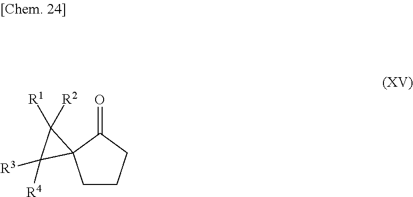

wherein R¹, R², R³, R⁴ each independently denotes a hydrogen atom, a halogen atom or a C₁-C₅ alkyl group.

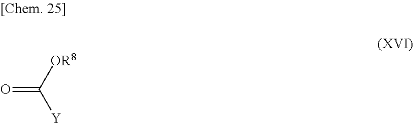

wherein R⁸ denotes a C₁-C₅ alkyl group; and Y denotes a C₁-C₅ alkoxy group or a halogen atom.

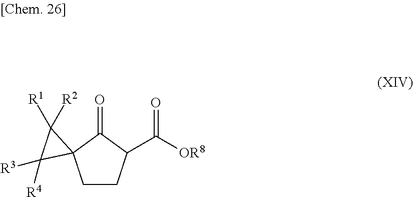

wherein R¹, R², R³, R⁴ each independently denotes a hydrogen atom, a halogen atom or a C₁-C₅ alkyl group; and R⁸ denotes a C₁-C₅ alkyl group.

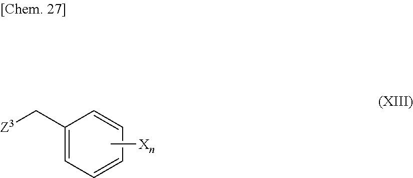

wherein Z³ denotes a halogen atom; X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different.

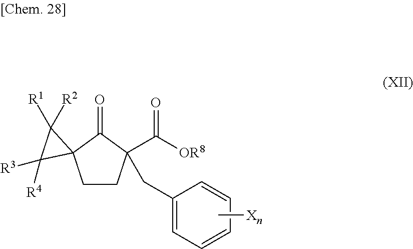

wherein X denotes a halogen atom, a C₁-C₅ alkyl group, a C₁-C₅haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and R⁸ denotes a C₁-C5 alkyl group.

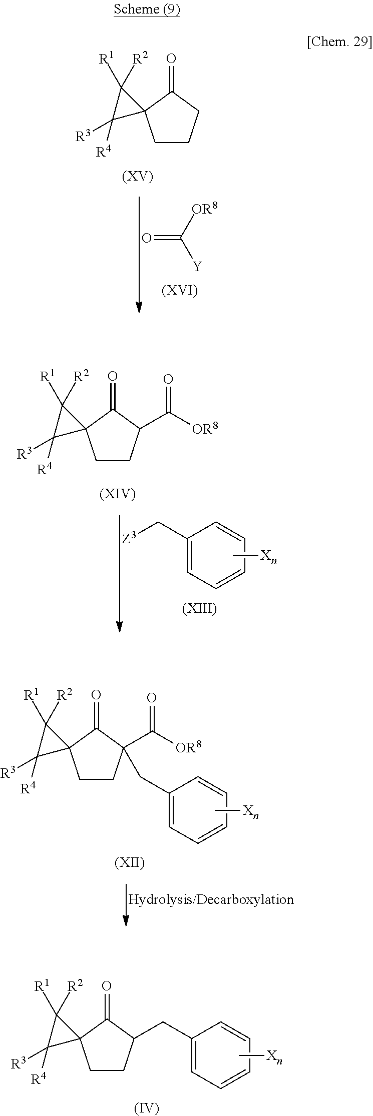

The reaction here to obtain Compound (XIV) by reacting Compound (XV) with Compound (XVI) in the presence of a base may be conducted in a solvent (step E), and if the Y is a C₁-C₅ alkoxy group then Compound (XVI) can be employed as a solvent.

The amount of Compound (XVI) employed is usually 0.5 to 20 moles, preferably 0.8 to 10 moles per mole of Compound (XV).

The base employed here preferably may for example be, but not limited to, an alkaline metal hydride such as sodium hydride and the like, and an alkaline metal alkoxide such as sodium methoxide, sodium ethoxide, potassium t-butoxide and the like. The amount of the base is usually 0.5 to 5 moles, preferably 0.8 to 2 moles per mole of Compound (X). The reaction temperature is usually 0 degrees C. to 250 degrees C., preferably room temperature to 150 degrees C., and the reaction time is usually 0.1 hour to several days, preferably 0.5 hours to 24 hours.

A cyclopentanone compound represented by Compound (XV) employed here can be synthesized by a method known in references.

The reaction in which a carbon-carbon bond is formed between the carbon atom to which an alkoxycarbonyl group of Compound (XIV) is bound and the carbon atom to which a halogen atom of Compound (XIII) is bound to give Compound (XII) (Step F) is conducted in a solvent in the presence of a base.

The amount of Compound (XIII) employed is usually 0.5 to 10 moles, preferably 0.8 to 5 moles per mole of Compound (XIV).

The base employed here preferably may for example be, but not limited to, an alkaline metal hydride such as sodium hydride and the like, and an alkaline metal carbonate such as sodium carbonate, potassium carbonate and the like.

The amount of the base is usually 0.5 to 5 moles, preferably 0.8 to 2 moles per mole of Compound (XIV).

The reaction temperature is usually 0 degrees C. to 250 degrees C., preferably room temperature to 150 degrees C., and the reaction time is usually 0.1 hour to several days, preferably 0.5 hours to 24 hours.

The reaction in which the hydrolysis/decarboxylation of the alkoxycarbonyl group of Compound (XII) obtained in the reaction described above is conducted (Step G) may be conducted in a solvent under a basic or acidic condition, preferably under a basic condition.

When the hydrolysis is conducted here under a basic condition, the base is usually an alkaline metal base such as sodium hydroxide, potassium hydroxide and the like. The solvent is usually water, or water combined for example with an alcohol.

When the hydrolysis is conducted here under an acidic condition, an inorganic acid such as hydrochloric acid, hydrobromic acid, sulfuric acid and the like is employed preferably as an acid catalyst, and the solvent is usually water, or water combined with an organic acid such as acetic acid.

The reaction temperature is usually 0 degrees C. to reflux temperature, preferably room temperature to reflux temperature. The reaction time is usually 0.1 hour to several days, preferably 0.5 hours to 24 hours.

As a preferred second synthesis method of a 2-(2-haloethyl)cyclopentanone compound represented by Formula (V) described above, a method comprising a step in which a ketoester compound represented by Formula (VII) and a 2-(lower alkoxy)alkyl halide compound represented by Formula (XVII) are reacted to obtain a 2-(lower alkoxy)alkylketoester compound represented by Formula (XVIII) (hereinafter referred to as Step H) followed by a step in which the alkoxycarbonyl group is hydrolyzed/decarboxylated while replacing the lower alkoxy group with a halogen atom to obtain a 2-(2-haloethyl)cyclopentanone compound represented by Formula (Va) (hereinafter referred to as Step I) may be exemplified (see Scheme (10)).

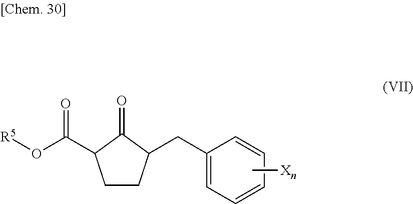

wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; and R⁵ denotes a C₁-C₄ alkyl group.

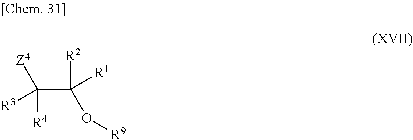

wherein R¹, R², R³, R⁴ each independently denotes a hydrogen atom, a halogen atom or a C₁-C₅ alkyl group; Z⁴ denotes a halogen atom; and R⁹ denotes a C₁-C₄ lower alkyl group.

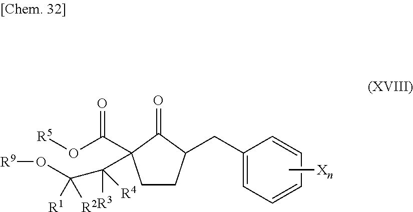

wherein X denotes a halogen atom, a C₁-C₅ alkyl group, a C₁-C₅ haloalkyl group, a C₁-C₅ alkoxyl group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; R⁵, R⁹ each independently denotes a C₁-C₄ lower alkyl group, and both of R⁵ and R⁹ are preferably methyl groups, ethyl groups, with methyl groups being preferred especially.

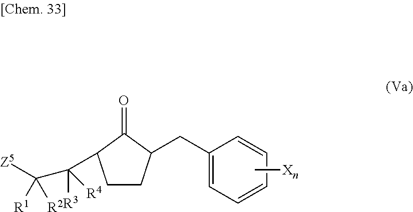

wherein X denotes a halogen atom, a C₁-C₅ alkyl group, a C₁-C₅ haloalkyl group, a C₁-C₅ alkoxyl group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and Z⁵ denotes a halogen atom, preferably a bromine atom, a chlorine atom, with a bromine atom being preferred especially.

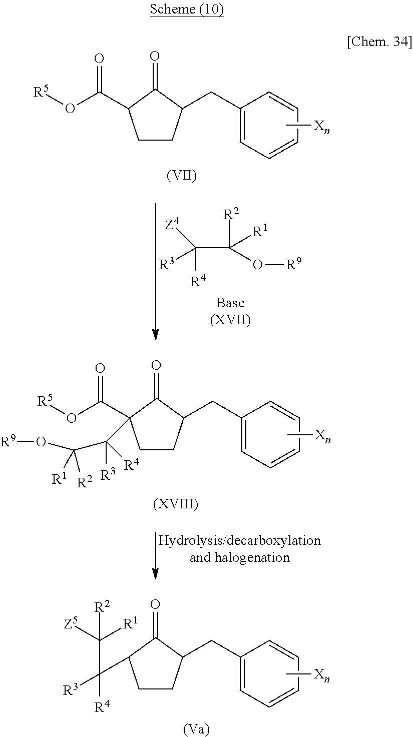

Step H is conducted by reacting the ketoester compound represented by Formula (VII) described above and the 2-(lower alkoxy)alkyl halide compound represented by Formula (XVII) described above in a solvent in the presence of a base.

The base employed here is not limited particularly and may for example be an alkaline metal hydride such as sodium hydride and the like, and an alkaline metal carbonate such as sodium carbonate, potassium carbonate and the like. The amount of the base is usually 0.5 to 5 moles, preferably 0.8 to 2 moles per mole of the ketoester compound represented by Formula (VII) described above.

The amount of the 2-(lower alkoxy)alkyl halide compound represented by Formula (XVII) described above employed is 0.5 to 10 moles, preferably 0.8 to 5 moles per mole of the ketoester compound represented by Formula (VII) described above.

The reaction temperature of Step H may appropriately be selected depending on the types of the solvent, the ketoester compound represented by Formula (VII) described above, the 2-(lower alkoxy)alkyl halide compound represented by Formula (XVII) described above, bases employed, and is preferably 0 degrees C. to 250 degrees C., more preferably room temperature to 150 degrees C. The reaction time may appropriately be selected depending on the types of the solvent, the ketoester compound represented by Formula (VII) described above, the 2-(lower alkoxy)alkyl halide compound represented by Formula (XVII) described above, bases employed, and is preferably 0.1 hour to several days, more preferably 0.5 hours to 24 hours.

Step I is conducted by subjecting the 2-(lower alkoxy)lated ketoester compound represented by Formula (VI) described above to hydrolysis/decarboxylation under an acidic condition while replacing the 2-(lower alkoxy) with a halogen atom.

The acid employed here is not limited particularly, and it is preferred to use a hydrohalic acid such as hydrobromic acid, hydrochloric acid and the like since the reaction system should have a halogen atom for replacing the 2-(lower alkoxy) with a halogen atom. The solvent employed is not limited particularly, and may be water with or without an organic acid such as acetic acid.

The reaction temperature of Step I may appropriately be selected depending on the solvent, the 2-(lower alkoxy)lated ketoester compound represented by Formula (XVIII) described above, and the acid catalyst employed, and is preferably 0 degrees C. to reflux temperature, more preferably room temperature to reflux temperature. The reaction time may appropriately be selected depending on the solvent, the 2-(lower alkoxy)lated ketoester compound represented by Formula (XVIII) described above, and the acid catalyst employed, and is preferably 0.1 hour to several days, more preferably 0.5 hours to 24 hours.

### C) Agro-Horticultural Agents and an Industrial Material Protecting Agents

Since an inventive 5-benzyl-4-azolylmethyl-4-spiro[2.4]heptanol derivative represented by Formula (I) described above has a 1,2,4-triazolyl group or imidazolyl group, it forms an acid addition salt of an inorganic or organic acid, as well as a metal complex. Accordingly, it can be used, while constituting a part of such an acid addition salt or a metal complex, as an active ingredient of an agro-horticultural agent and an industrial material protecting agent.

On the other hand, the 5-benzyl-4-azolylmethyl-4-spiro[2.4]heptanol derivative represented by Formula (I) has at least two asymmetric carbon atom. Accordingly, it may exist as a mixture of stereoisomers, as a mixture of optical isomers, as either stereoisomer or optical isomer, the invention is not limited to any of the mixture of stereoisomers, the mixture of optical isomers, the stereoisomer or the optical isomer. Thus, at least one of these stereoisomers or optical isomers can be used as an active ingredient of an agro-horticultural agent and an industrial material protecting agent.

The usefulness of a 5-benzyl-4-azolylmethyl-4-spiro[2.4]heptanol derivative represented by Formula (I) according to the invention as an active ingredient of an agro-horticultural agent and an industrial material protecting agent is explained below.

Compound (I) of the invention exhibits a controlling effect on abroad range of plant diseases. Such diseases are exemplified below. Soybean rust (Phakopsora pachyrhizi, Phakopsora meibomiae), rice blast (Pyricularia oryzae), rice brown spot (Cochliobolus miyabeanus), rice leaf blight (Xanthomonas oryzae), rice sheath blight (Rhizoctonia solani), rice stem rot (Helminthosporium sigmoideun), rice bakanae disease (Gibberella fujikuroi), rice root rots blight (Pythium aphanidermatum), apple powdery mildew (Podosphaera leucotricha), apple scab (Venturia inaequalis), apple blossom blight (Monilinia mali), apple alternaria blotch (Alternaria alternata), apple valsa canker (Valsa mali), pear black spot (Alternaria kikuchiana), pear powdery mildew (Phyllactinia pyri), pear rust (Gymnosporangium asiaticum), pear scab (Venturia nashicola), grape powdery mildew (Uncinula necator), grape downy mildew (Plasmopara viticola), grape ripe rot (Glomerella cingulata), barley powdery mildew (Erysiphe graminis f. sp hordei), barley stem rust (Puccinia graminis), barley stripe rust (Puccinia striiformis), barley stripe (Pyrenophora graminea), barley scald (Rhynchosporium secalis), wheat powdery mildew (Erysiphe graminis f. sp tritici), wheat leaf rust (Puccinia recondita), wheat stripe rust (Puccinia striiformis), wheat eye spot (Pseudocercosporella herpotrichoides), wheat fusarium blight (Fusarium graminearum, Microdochium nivale), wheat stagonospora blotch (Phaeosphaeria nodorum), wheat septoria bloth (Septoria tritici), gourd powdery mildew (Sphaerotheca fuliginea), gourd anthracnose (Colletotrichum lagenarium), cucumber downy mildew (Pseudoperonospora cubensis), cucumber gray mold (Phytophthora capsici), tomato powdery mildew (Erysiphe cichoracearum), tomato early blight (Alternaria solani), eggplant powdery mildew (Erysiphe cichoracearum), strawberry powdery mildew (Sphaerotheca humuli), tobacco powdery mildew (Erysiphe cichoracearum), sugar beet cercpspora leaf spot (Cercospora beticola), maize smut (Ustillaga maydis), plum brown rot (Monilinia fructicola), various plants-affecting gray mold (Botrytis cinerea), sclerotinia rot (Sclerotinia sclerotiorum) and the like may be exemplified.

Furthermore, the inventive Compound (I) exhibits yield-increasing effects or quality-improving effects on a broad range of crops and horticultural plants. Such crops may for example be those listed below. Wheat, barley, oats, rice, rapeseed, sugarcane, corn, maize, soybean, pea, peanut, sugar beet, cabbage, garlic, radish, carrot, apple, pear, citric fluits such as mandarin, orange, lemon and the like, peach, cherry, avocado, mango, papaya, red pepper, cucumber, melon, strawberry, tobacco, tomato, eggplant, turf, chrysanthemum, azalea, other ornamental plants.

Moreover, the inventive Compound (I) exhibits an excellent ability of protecting an industrial material from a broad spectrum of hazardous microorganisms which invade such a material. Examples of such microorganisms are listed below.

Paper/pulp deteriorating microorganisms (including slime-forming microorganisms) such as Aspergillus sp., Trichoderma sp., Penicillium sp., Geotrichum sp., Chaetomium sp., Cadophora sp., Ceratostomella sp., Cladosporium sp., Corticium sp., Lentinus sp., Lezites sp., Phoma sp., Polysticus sp., Pullularia sp., Stereum sp., Trichosporium sp., Aerobacter sp., Bacillus sp., Desulfovibrio sp., Pseudomonas sp., Flavobacterium sp. and Micrococcus sp.; fiber-deteriorating microorganisms such as Aspergillus sp., Penicillium sp., Chaetomium sp., Myrothecium sp., Curvularia sp., Gliomastix sp., Memnoniella sp., Sarcopodium sp., Stachybotrys sp., Stemphylium sp., Zygorhynchus sp., Bacillus sp. and Staphylococcus sp.; lumber-deteriorating microorganisms such as Tyromyces palustris, Coriolus versicolor, Aspergillus sp., Penicillium sp., Rhizopus sp., Aureobasidium sp., Gliocladium sp., Cladosporium sp., Chaetomium sp. and Trichoderma sp.; leather-deteriorating microorganisms such as Aspergillus sp., Penicillium sp., Chaetomium sp., Cladosporium sp., Mucor sp., Paecilomyces sp., Pilobus sp., Pullularia sp., Trichosporon sp. and Tricothecium sp.; rubber/plastic-deteriorating microorganisms such as Aspergillus sp., Penicillium sp., Rhizopus sp., Trichoderma sp., Chaetomium sp., Myrothecium sp., Streptomyces sp., Pseudomonas sp., Bacillus sp., Micrococcus sp., Serratia sp., Margarinomyces sp. and Monascus sp.; paint-deteriorating microorganisms such as Aspergillus sp., Penicillium sp., Cladosporium sp., Aureobasidium sp., Gliocladium sp., Botryodiplodia sp., Macrosporium sp., Monilia sp., Phoma sp., Pullularia sp., Sporotrichum sp., Trichoderma sp., Bacillus sp., Proteus sp., Pseudomonas sp. and Serratia sp.

While an inventive compound may be applied, as an active ingredient of an agro-horticultural pesticide, alone without any other components, it is usually combined with a solid carrier, a liquid carrier, a surfactant, other formulation auxiliary agents to be formulated into various formulations such as a powder, wettable powder, granule, emulsifiable concentrate and the like. Such a formulation is formulated so that it contains the inventive compound as an active ingredient in an amount of 0.1 to 95% by weight, preferably 0.5 to 90% by weight, more preferably 2 to 80% by weight. Examples of carriers, diluents and surfactants employed as formulation auxiliary agents are solid carriers including talc, kaolin, bentonite, diatomaceous earth, white carbon, clay and the like, liquid carriers including water, xylene, toluene, chlorobenzene, cyclohexane, cyclohexanone, dimethyl sulfoxide, dimethyl formamide, alcohols and the like. The surfactant may appropriately selected for an intended effect, and the emulsifier may for example be polyoxiethylene alkylaryl ether, polyoxyethylene sorbitan monolaurate. The dispersing agent may for example be lignin sulfonate, dibutylnaphthalene sulfonate and the like, and the wetting agent may for example be an alkyl sulfonate, alkylphenyl sulfonate and the like. The formulation described above may be used as it is, or used as being diluted in a diluent such as water to a certain concentration. The concentration of the inventive compound when used as being diluted is preferably 0.001 to 1.0%. The amount of the inventive compound for 1 ha of the agro-horticultural field such as a farm, paddy field, orchard, greenhouse and the like is 20 to 5000 g, more preferably 50 to 2000 g. Since these concentration and amount to be used may vary depending on the dosage form, time of use, method of use, place of use, subject crop and the like, it is a matter of course that they can be increased or decreased regardless of the ranges mentioned above. In addition, the inventive compound can be used in combination with other active ingredients, such as fungicides, bactericides, insecticides, acaricides, herbicides and the like.

For example, by mixing with the agents listed below, the performance of an agro-horticultural agent can be enhanced.

### <Fungicides/Bactericides>

Acibenzolar-S-methyl, 2-phenylphenol (OPP), azaconazole, azoxystrobin, amisulbrom, bixafen, benalaxyl, benomyl, benthiavalicarb-isopropyl, bicarbonate, biphenyl, bitertanol, blasticidin-S, borax, Bordeaux mixture, boscalid, bromuconazole, bronopol, bupirimate, sec-butylamine, calcium polysulphide, captafol, captan, carbendazim, carboxin, carpropamid, quinomethionate, chloroneb, chloropicrin, chlorothalonil, chlozolinate, cyazofamid, cyflufenamid, cymoxanil, cyproconazole, cyprodinil, dazomet, debacarb, dichlofluanid, diclocymet, diclomezine, dicloran, diethofencarb, difenoconazole, diflumetorim, dimethomorph, dimoxystrobin, diniconazole, dinocap, diphenylamine, dithianon, dodemorph, dodine, edifenphos, epoxiconazole, ethaboxam, ethoxyquin, etridiazole, enestroburin, famoxadone, fenamidone, fenarimol, fenbuconazole, fenfuram, fenhexamid, fenoxanil, fenpiclonil, fenpropidin, fenpropimorph, fentin, ferbam, ferimzone, fluazinam, fludioxonil, flumorph, fluoroimide, fluoxastrobin, fluquinconazole, flusilazole, flusulfamide, flutolanil, flutriafol, folpet, fosetyl-Al, fuberidazole, furalaxyl, furametpyr, fluopicolide, fluopyram, guazatine, hexachlorobenzene, hexaconazole, hymexazol, imazalil, imibenconazole, iminoctadine, ipconazole, iprobenfos, iprodione, iprovalicarb, isoprothiolane, isopyrazam, isotianil, kasugamycin, copper preparations, such as: copper hydroxide, copper naphthenate, copper oxychloride, copper sulphate, copper oxide, oxine copper, kresoxim-methyl, mancopper, mancozeb, maneb, mandipropamid, mepanipyrim, mepronil, metalaxyl, metconazole, metiram, metominostrobin, mildiomycin, myclobutanil, nitrothal-isopropyl, nuarimol, ofurace, oxadixyl, oxolinic acid, oxpoconazole, oxycarboxin, oxytetracycline, pefurazoate, orysastrobin, penconazole, pencycuron, penthiopyrad, pyribencarb, fthalide, picoxystrobin, piperalin, polyoxin, probenazole, prochloraz, procymidone, propamocarb, propiconazole, propineb, proquinazid, prothioconazole, pyraclostrobin, pyrazophos, pyrifenox, pyrimethanil, pyroquilon, quinoxyfen, quintozene, silthiopham, simeconazole, spiroxamine, Sulfur and sulfur formulations, tebuconazole, tecloftalam, tecnazen, tetraconazole, thiabendazole, thifluzamide, thiophanate-methyl, thiram, thiadinil, tolclofos-methyl, tolylfluanid, triadimefon, triadimenol, triazoxide, tricyclazole, tridemorph, trifloxystrobin, triflumizole, triforine, triticonazole, validamycin, vinclozolin, zineb, ziram, zoxamide and the like.

### <Insecticides/Acaricides/Nematocides>

Abamectin, acephate, acrinathrin, alanycarb, aldicarb, allethrin, amitraz, avermectin, azadirachtin, azamethiphos, azinphos-ethyl, azinphos-methyl, azocyclotin, Bacillus firmus, Bacillus subtilis, Bacillus thuringiensis, bendiocarb, benfuracarb, bensultap, benzoximate, bifenazate, bifenthrin, bioallethrin, bioresmethrin, bistrifluoron, buprofezin, butocarboxim, butoxycarboxim, cadusafos, carbaryl, carbofuran, carbosulfan, cartap, CGA50439, chlordane, chlorethoxyfos, chlorphenapyr, chlorfenvinphos, chlorfluazuron, chlormephos, chlorpyrifos, chlorpyrifos methyl, chromafenozide, clofentezine, clothianidin, chlorantraniliprole, coumaphos, cryolite, cyanophos, cycloprothrin, cyfluthrin, cyhalothrin, cyhexatin, cypermethrin, cyphenothrin, cyromazine, cyenopyrafen, DCIP, DDT, deltamethrin, demeton-S-methyl, diafenthiuron, diazinon, dichlorophen, dichloropropene, dichlorvos, dicofol, dicrotophos, dicyclanil, diflubenzuron, dimethoate, dimethylvinphos, dinobuton, dinotefuran, emamectin, endosulfan, EPN, esfenvalerate, ethiofencarb, ethion, ethiprole, ethofenprox, ethoprophos, etoxazole, famphur, fenamiphos, fenazaquin, fenbutatin oxide, fenitrothion, fenobucarb, fenothiocarb, fenoxycarb, fenpropathrin, fenpyroximate, fenthion, fenvalerate, fipronil, flonicamid, fluacrypyrim, flucycloxuron, flucythrinate, flufenoxuron, flumethrin, fluvalinate, flubendiamide, formetanate, fosthiazate, halfenprox, furathiocarb, halofenozide, gamma-HCH, heptenophos, hexaflumuron, hexythiazox, hydramethylnon, imidacloprid, imiprothrin, indoxacarb, isoprocarb, isoxathion, lufenuron, malathion, mecarbam, metam, methamidophos, methidathion, methiocarb, methomyl, methoprene, methothrin, methoxyfenozide, metolcarb, milbemectin, monocrotophos, naled, nicotine, nitenpyram, novaluron, noviflumuron, omethoate, oxamyl, oxydemethon methyl, parathion, permethrin, phenthoate, phorate, phosalone, phosmet, phosphamidon, phoxim, pirimicarb, pirimiphos-methyl, profenofos, propoxur, prothiophos, pymetrozin, pyrachlophos, pyrethrin, pyridaben, pyridalyl, pyrimidifen, pyriproxifen, pyrifluquinazon, pyriprole, quinalphos, silafluofen, spinosad, spirodiclofen, spiromesifen, spirotetramat, sulfluramid, sulphotep, SZI-121, tebufenozid, tebufenpyrad, tebupirimphos, teflubenzuron, tefluthrin, temephos, terbufos, tetrachlorvinphos, thiacloprid, thiamethoxam, thiodicarb, thiofanox, thiometon, tolfenpyrad, tralomethrin, tralopyril, triazamate, triazophos, trichlorfon, triflumuron, vamidothion, XMC, xylylcarb.

### <Plant Growth Regulators>

Ancymidol, 6-benzylaminopurine, paclobutrazol, diclobutrazole, mepiquat chloride, uniconazole.

While an inventive compound (I) may be applied, as an active ingredient of an industrial material protecting agent, alone without any other components, it is generally dissolved or dispersed in a suitable liquid carrier, or mixed with a solid carrier, and combined if necessary with emulsifier, dispersing agent, spreading agent, penetrating agent, wetting agent, stabilizer and the like and formulated into a dosage form such as wettable powder, powder, granule, tablet, paste, suspension, spray and the like. It may also be supplemented with other fungicides, bactericides, insecticides, deterioration-preventing agent and the like.

The liquid carrier may be any liquid as long as it does not react with an active ingredient, and may be selected from water, alcohols (for example, methyl alcohol, ethyl alcohol, ethylene glycol, cellosolve and the like), ketones (for example, acetone, methylethylketone and the like), ethers (for example, dimethyl ether, diethyl ether, dioxane, tetrahydrofuran and the like), aromatic hydrocarbons (for example, benzene, toluene, xylene, methylnaphthalene and the like), aliphatic hydrocarbons (for example, gasoline, kerosene, paraffin oil, machine oil, fuel oil and the like), acid amides (for example, dimethyl formamide, N-methylpyrrolidone and the like), halogenated hydrocarbons (for example, chloroform, carbon tetrachloride and the like), esters (for example, acetic acid ethyl ester, fatty acid glycerin ester and the like), nitriles (for example, acetonitrile and the like), and dimethyl sulfoxide and the like. The solid carrier may for example be a microparticle or a granule of kaolin clay, bentonite, acid clay, pyrophylite, talc, diatomaceous earth, calcite, urea, ammonium sulfate. The emulsifiers and the dispersing agents may for example be soaps, alkyl sulfonates, alkylaryl sulfonates, dialkyl sulfosuccinates, quaternary ammonium salts, oxyalkylamines, fatty acid esters, polyalkylene oxide-based, anhydrosorbitol-based surfactants.

When the inventive compound (I) is contained as an active ingredient in a formulation, it is added in such an amount that the concentration becomes 0.1 to 99.9% by weight, although the content may vary depending on the dosage form and the purpose of use. Upon being used practically, it is combined appropriately with a solvent, diluent, extender and the like so that the treatment concentration is usually 0.005 to 5% by weight, preferably 0.01 to 1% by weight.

### EXAMPLES

The invention is embodied below with referring to Production Examples, Formulation Examples, and Experiment Examples. The invention is not restricted to the following Production Examples, Formulation Examples, and Experiment Examples unless departing from its scope.

### Production Example 1

### Synthesis of 5-(4-chlorobenzyl)-4-(1H-1,2,4-triazol-1-ylmethyl)-4-spiro[2.4]heptanol (Compound No. I-1(Compound (I), X=4-Cl, n=1, R¹=H, R²=H, R³=H, R⁴=H, Isomer Form C) and Compound No. I-2(Compound (I), X=4-Cl, n=1, R¹=H, R²=H, R³=H, R⁴=H, Isomer Form T))

### (1) Synthesis of Intermediate, 9-(4-chlorobenzyl)-1-oxadispiro[2.0.2.3]nonane (Compound (II), X=4-Cl, n=1, R¹=H, R²=H, R³=H, R⁴=H)

Under nitrogen flow, 606 sodium hydride (246 mg, 6.1 mmol) was washed with hexane, and then suspended in DMSO (2 ml), and trimethylsulfonium iodide (1.28 g, 6.1 mmol) was added. After stirring at room temperature for 5 minutes, while cooling with ice, a DMSO (2 ml) solution of 5-(4-chlorobenzyl)-4-spiro[2.4]heptanone (Compound (IV), X=4-Cl, n=1, R¹=H, R²=H, R³=H, R⁴=H) (961 mg, 4.1 mmol) was added, and stirring was continued at room temperature for 16 hours. The reaction solution was poured into iced water, and extracted with ethyl acetate. The organic layer was washed with water, saturated brine, and dried over anhydrous sodium sulfate. The solvent was distilled off under reduced pressure, and a crude title compound was obtained.

Crude product: 926 mg, Rough yield: 90%, Tan oil.

¹H-NMR (CDCl₃) delta 0.3-0.6 (m, 4H), 1.5-1.8 (m, 2H), 1.8-2.0 (m, 2H), 2.38 (d, J=4.3 Hz, 1H), 2.4-2.5 (m, 2H), 2.56 (d, J=4.3 Hz, 1H), 2.77 (dd, J=13.2, 5.7 Hz, 1H), 7.11 (d, J=8.4 Hz, 2H), 7.23 (d, J=8.4 Hz, 2H).

### (2) Synthesis of 5-(4-chlorobenzyl)-4-(1H-1,2,4-triazol-1-ylmethyl)-4-spiro[2.4]heptanol (Compound No. I-1(Compound (I), X=4-Cl, n=1, R¹=H, R²=H, R³=H, R⁴=H, Isomer Form C) and Compound No. I-2(Compound (I), X=4-Cl, n=1, R¹=H, R²=H, R³=H, R⁴=H, Isomer Form T))

Under nitrogen flow, 60% sodium hydride (149 mg, 3.7 mmol) was washed with hexane, and then suspended in anhydrous DMF (1 ml), while cooling with ice, 1H-1,2,4-triazole (257 mg, 3.7 mmol) was added. After stirring at room temperature for 5 minutes, an anhydrous DMSO (2 ml) solution of 9-(4-chlorobenzyl)-1-oxadispiro[2.0.2.3]nonane (Compound (II), X=4-Cl, n=1, R¹=H, R²=H, R³=H, R⁴=H) (926 mg, 3.7 mmol) was added, and stirred for 3 hours at 120 degrees C. The reaction solution was poured into an iced water, and extracted with ethyl acetate. The organic layer was washed with water, saturated brine, and dried over anhydrous sodium sulfate. The solvent was distilled off under reduced pressure, and the resultant crude product was purified by silica gel column chromatography (eluent, hexane-ethyl acetate, 1:1) to obtain the title compound.

Compound No. I-1

Product: 141 mg, Yield: 11%, White crystal, melting point: 85.5-87.0 degrees C.

¹H-NMR (CDCl₃) delta 0.33 (ddd, J=9.4, 6.0, 4.1 Hz, 1H), 0.5-0.6 (m, 2H), 0.80 (ddd, J=10.0, 6.0, 4.1 Hz, 1H), 1.5-1.6 (m, 2H), 1.6-1.8 (m, 2H), 2.0-2.1 (m, 1H), 2.33 (dd, J=13.7, 10.3 Hz, 1H), 2.40 (dd, J=13.7, 4.9 Hz, 1H), 3.16 (bs, 1H), 4.05 (d, J=14.2 Hz, 1H), 4.19 (d, J=14.2 Hz, 1H), 6.95 (d, J=8.4 Hz, 2H), 7.19 (d, J=8.4 Hz, 2H), 7.97 (s, 1H), 8.16 (s, 1H).

Compound No. I-2

Product: 68 mg, Yield: 5%, White crystal, melting point: 166-167 degrees C.

¹H-NMR (CDCl₃) delta −0.31 (ddd, J=10.2, 5.9, 5.1 Hz, 1H), 0.09 (ddd, J=9.5, 5.9, 4.0 Hz, 1H), 0.24 (ddd, J=9.5, 5.9, 5.1 Hz, 1H), 0.60 (ddd, J=10.2, 5.9, 4.0 Hz, 1H), 1.3-1.5 (m, 2H), 1.8-2.0 (m, 2H), 2.3-2.4 (m, 2H), 3.07 (d, J=9.5 Hz, 1H), 3.29 (5, 1H), 4.24 (d, J=14.0 Hz, 1H), 4.32 (d, J=14.0 Hz, 1H), 7.11 (d, J=8.4 Hz, 2H), 7.25 (d, J=8.4 Hz, 2H), 7.98 (s, 1H), 8.14 (s, 1H).

### Production Example 2

### Synthesis of 5-(4-chlorobenzyl)-4-(1H-1,2,4-triazol-1-ylmethyl)-4-spiro[2.4]heptanol (Compound No. I-1(Compound (I), X=4-Cl, n=1, R¹=H, R²=H, R³=H, R⁴=H, Isomer Form C) and Compound No. I-2(Compound (I), X=4-Cl, n=1, R¹=H, R²=H, R³=H, R⁴=H, Isomer Form T))

Under argon flow, anhydrous NMP180 ml was combined with triazole sodium salt (40.0 g, 505.2 mmol) was added, and heated to about 120 degrees C.

5-(4-Chlorobenzyl)-4-spiro[2.4]heptanone (Compound (IV), X=4-Cl, n=1, R¹=H, R²=H, R³=H, R⁴=H) (94.0 g, 400 mmol) was added together with NMP (20 ml). At about 120 degrees C. and over about 3 hours, t-BuONa (23.14 g, 240 mmol) and trimethyl sulfoxonium bromide (88.4 g, 511 mmol) were added intermittently in portions, independently. After completing the addition followed by stirring at the same temperature for 1 hour, water was added and extraction was made with water. The organic layer was washed with water, saturated brine, and dried over anhydrous sodium sulfate. The crude title compound was obtained. A quantitative analysis of the crude title compound revealed the production at the yield shown below.

Compound No. I-1

Product: 53.68 g, Yield: 42%

Compound No. I-2

Product: 28.71 g, Yield: 23%

### Production Example 3

### Synthesis of 5-(3-chlorobenzyl)-4-(1H-1,2,4-triazol-1-ylmethyl)-4-spiro[2.4]heptanol (Compound No. I-3(Compound (I), X=3-Cl, n=1, R¹=H, R²=H, R³=H, R⁴=H, Isomer Form C) and Compound No. I-4(Compound (I), X=3-Cl, n=1, R¹=H, R²=H, R³=H, R⁴=H, Isomer Form T))

### (1) Synthesis of Intermediate, 9-(3-chlorobenzyl)-1-oxadispiro[2.0.2.3]nonane (Compound (II), X=3-Cl, n=1, R¹=H, R²=H, R³=H, R⁴=H)

Under nitrogen flow, 60% sodium hydride (189 mg, 4.7 mmol) was washed with hexane, and then suspended in DMSO (3 ml), trimethylsulfonium iodide (983 mg, 4.7 mmol) was added. After stirring at room temperature for 5 minutes, while cooling with ice, a DMSO (2 ml) solution of 5-(3-chlorobenzyl)-4-spiro[2.4] heptanone (Compound (IV), X=3-Cl, n=1, R¹=H, R²=H, R³=H, R⁴=H) (739 mg, 3.2 mmol) was added, and stirring was continued for 6.5 hours at room temperature. The reaction solution was poured into an iced water, and extracted with ethyl acetate. The organic layer was washed with water, saturated brine, and dried over anhydrous sodium sulfate. The solvent was distilled off under reduced pressure, and a crude title compound was obtained.

Crude product: 748 mg, Rough yield: 95%, Yellow oil.

### (2) Synthesis of 5-(3-chlorobenzyl)-4-(1H-1,2,4-triazol-1-ylmethyl)-4-spiro[2.4]heptanol (Compound No. I-3(Compound (I), X=3-Cl, n=1, R¹=H, R²=H, R³=H, R⁴=H, Isomer Form C) and Compound No. I-4(Compound (I), X=3-Cl, n=1, R¹=H, R²=H, R³=H, R⁴=H, Isomer Form T))

Under nitrogen flow, 60% sodium hydride (120 mg, 3.0 mmol) was washed with hexane, and then suspended in anhydrous DMF (2 ml), and then while cooling with ice 1H-1,2,4-triazole (208 mg, 3.0 mmol) was added. After stirring at room temperature for 5 minutes, an anhydrous DMF (2 ml) solution of 9-(3-chlorobenzyl)-1-oxadispiro[2.0.2.3]nonane (Compound (II), X=3-Cl, n=1, R¹=H, R²=H, R³=H, R⁴=H) (748 mg, 3.0 mmol) was added, and stirring was continued for 4 hours at 120 degrees C. The reaction solution was poured into an iced water, and extracted with ethyl acetate. The organic layer was washed with water, saturated brine, and dried over anhydrous sodium sulfate. The solvent was distilled off under reduced pressure, and the resultant crude product was purified by silica gel column chromatography (eluent, hexane-ethyl acetate, 1:1) to obtain the title compound.

Compound No. I-3

Product: 159 mg, Yield: 16%, Pale tan crystal, melting point: 84.5-85.5 degrees C.

¹H-NMR (CDCl₃) delta 0.33 (ddd, J=9.4, 6.0, 4.1 Hz, 1H), 0.49 (ddd, J=9.4, 6.0, 4.8 Hz, 1H), 0.55 (ddd, J=9.9, 6.0, 4.8 Hz, 1H), 0.79 (ddd, J=9.9, 6.0, 4.1 Hz, 1H), 1.5-1.6 (m, 2H), 1.6-1.8 (m, 2H), 2.0-2.1 (m, 1H), 2.35 (dd, J=13.7, 10.7 Hz, 1H), 2.43 (dd, J=13.7, 4.6 Hz, 1H), 3.17 (s, 1H), 4.06 (d, J=14.2 Hz, 1H), 4.20 (d, J=14.2 Hz, 1H), 6.89 (dt, J=6.7, 1.9 Hz, 1H), 7.03 (bs, 1H), 7.1-7.2 (m, 2H), 7.98 (s, 1H), 8.16 (s, 1H).

Compound No. I-4

Product: 209 mg, Yield: 21%, White crystal, melting point: 120-121 degrees C.

¹H-NMR (CDCl₃) delta −0.31 (ddd, J=10.2, 5.9, 5.1 Hz, 1H), 0.09 (ddd, J=9.5, 5.9, 4.1 Hz, 1H), 0.25 (ddd, J=9.5, 5.9, 5.1 Hz, 1H), 0.60 (ddd, J=10.2, 5.9, 4.1 Hz, 1H), 1.4-1.6 (m, 2H), 1.9-2.0 (m, 2H), 2.30 (d, J=9.9 Hz, 1H), 2.3-2.4 (m, 1H), 3.09 (d, J=9.9 Hz, 1H), 3.33 (s, 1H), 4.24 (d, J=14.0 Hz, 1H), 4.32 (d, J=14.0 Hz, 1H), 7.06 (dt, J=7.0, 1.6 Hz, 1H), 7.1-7.3 (m, 3H), 7.98 (s, 1H), 8.15 (s, 1H).

### Production Example 4

### Synthesis of 5-(4-fluorobenzyl)-4-(1H-1,2,4-triazol-1-ylmethyl)-4-spiro[2.4]heptanol (Compound No. I-7(Compound (I), X=4-F, n=1, R¹=H, R²=H, R³=H, R⁴=H, Isomer Form C) and Compound No. I-8(Compound (I), X=4-F, n=1, R¹=H, R²=H, R³=H, R⁴=H, Isomer Form T))

### (1) Synthesis of 9-(4-fluorobenzyl)-1-oxadispiro[2.0.2.3]nonane (Compound (II), X=4-F, n=1, R¹=H, R²=H, R³=H, R⁴=H)

Under nitrogen flow, 60% sodium hydride (178 mg, 4.5 mmol) was washed with hexane, and then suspended in DMSO (3 ml), and trimethylsulfonium iodide (929 mg, 4.5 mmol) was added. After stirring at room temperature for 5 minutes, while cooling with ice, a DMSO (3 ml) solution of 5-(4-fluorobenzyl)-4-spiro[2.4]heptanone (Compound (IV), X=4-F, n=1, R¹=H, R²=H, R³=H, R⁴=H) (649 mg, 3.0 mmol) was added, and stirring was continued at room temperature for 12 hours. The reaction solution was poured into an iced water, and extracted with ethyl acetate. The organic layer was washed with water, saturated brine, and dried over anhydrous sodium sulfate. The solvent was distilled of under reduced pressure, and a crude title compound was obtained.

Crude product: 680 mg, Rough yield: 99%, Pale yellow oil.

### (2) Synthesis of 5-(4-fluorobenzyl)-4-(1H-1,2,4-triazol-1-ylmethyl)-4-spiro[2.4]heptanol (Compound No. I-7(Compound (I), X=4-F, n=1, R¹=H, R²=H, R³=H, R⁴=H, Isomer Form C) and Compound No. I-8(Compound (I), X=4-F, n=1, R¹=H, R²=H, R³=H, R⁴=H, Isomer Form T))

Under nitrogen flow, 60% sodium hydride (117 mg, 2.9 mmol) was washed with hexane, and then suspended in anhyrdous DMF (2 ml), while cooling with ice, 1H-1,2,4-triazole (202 mg, 2.9 mmol) was added. After stirring at room temperature for 5 minutes, an anhydrous DMF (2 ml) solution of 9-(4-fluorobenzyl)-1-oxadispiro[2.0.2.3]nonane (Compound (II), X=4-F, n=1, R¹=H, R²=H, R³=H, R⁴=H) (680 mg, 2.9 mmol) was added, and stirring was continued at 120 degrees C. for 4.5 hours. The reaction solution was poured into an iced water, and extracted with ethyl acetate. The organic layer was washed with water, saturated brine, and dried over anhydrous sodium sulfate. The solvent was distilled off under reduced pressure, and the resultant crude product was purified by silica gel column chromatography (eluent, hexane-ethyl acetate, 1:1) to obtain the title compound.

Compound No. I-7

Product: 119 mg, Yield: 13%, White crystal, melting point: 89-90 degrees C.

¹H-NMR (CDCl₃) delta 0.33 (ddd, J=9.4, 6.0, 4.1 Hz, 1H), 0.50 (ddd, J=9.4, 6.0, 4.8 Hz, 1H), 0.56 (ddd, J=10.0, 6.0, 4.8 Hz, 1H), 0.80 (ddd, J=10.0, 6.0, 4.1 Hz, 1H), 1.5-1.6 (m, 2H), 1.6-1.8 (m, 2H), 2.0-2.1 (m, 1H), 2.34 (dd, J=13.7, 10.3 Hz, 1H), 2.40 (dd, J=13.7, 5.1 Hz, 1H), 3.09 (s, 1H), 4.05 (d, J=14.2 Hz, 1H), 4.19 (d, J=14.2 Hz, 1H), 6.8-7.0 (m, 4H), 7.98 (s, 1H), 8.16 (s, 1H).

Compound No. I-8

Product: 27 mg, Yield: 3%, White crystal, melting point: 139-140 degrees C.

¹H-NMR (CDCl₃) delta −0.31 (dt, J=10.0, 5.6 Hz, 1H), 0.09 (ddd, J=9.5, 5.7, 4.1 Hz, 1H), 0.2-0.3 (m, 1H), 0.60 (ddd, J=10.0, 5.7, 4.1 Hz, 1H), 1.5-1.6 (m, 2H), 1.9-2.0 (m, 2H), 2.3-2.4 (m, 2H), 3.0-3.1 (m, 1H), 4.24 (d, J=14.0 Hz, 1H), 4.33 (d, J=14.0 Hz, 1H), 6.96 (d, J=8.4 Hz, 1H), 6.98 (d, J=8.4 Hz, 1H), 7.12 (d, J=8.4 Hz, 1H), 7.13 (d, J=8.4 Hz, 1H), 7.98 (s, 1H), 8.15 (s, 1H).

### Production Example 5

### Synthesis of 5-(4-chlorobenzyl)-1,1-dimethyl-4-(1H-1,2,4-triazol-1-ylmethyl)-4-spiro[2.4]heptanol (Compound No. I-101(Compound (I), X=4-Cl, n=1, R¹=H, R²=H, R³=Me, R⁴=Me, Isomer Form C) and Compound No. I-151(Compound (I), X=4-Cl, n=1, R¹=Me, R²=Me, R³=H, R⁴=H, Isomer Form C))

### (1) Synthesis of 9-(4-chlorobenzyl)-5,5-dimethyl-1-oxadispiro[2.0.2.3]nonane (Mixture of Compound (II), X=4-Cl, n=1, R¹=H, R²=H, R³=Me, R⁴=Me and Compound (II), X=4-Cl, n=1, R¹=Me, R²=Me, R³=H, R⁴=H)

Under argon flow, samarium (powder, −20 mesh, SOEGAWA KAGAKU) (697 mg, 4.6 mmol) was suspended in anhydrous THF (3 ml), a trace amount of iodine was added, and then 1,2-diiodoethane (652 mg, 2.3 mmol) was added, and stirring was continued for 1 hour at 0 degrees C. While cooling with ice, a solution of 5-(4-chlorobenzyl)-1,1-dimethyl-4-spiro[2.4]heptanone (Compound (IV), X=4-Cl, n=1, R¹=H, R²=H, R³=Me, R⁴=Me (304 mg, 1.2 mmol) and diiodomethane (316 mg, 1.2 mmol) dissolved in anhydrous THF (1 ml) was added dropwise over 5 minutes. After stirring for 30 minutes at 0 degrees C., while cooling with ice, 10% aqueous solution of sodium hydroxide (1 ml) was added dropwise and portionwise, and then stirring was continued further for 1.5 hours at 0 degrees C. Solid materials were removed by filtration with aspiration, and extraction was made with ethyl acetate. The organic layer was washed with water, saturated brine, and dried over anhydrous sodium sulfate. The solvent was distilled off under reduced pressure, and a crude title compound was obtained.

Crude product: 314 mg, Rough yield: 92%, Pale yellow oil.

### (2) Synthesis of 5-(4-chlorobenzyl)-1,1-dimethyl-4-(1H-1,2,4-triazol-1-ylmethyl)-4-spiro[2.4]heptanol (Compound No. I-101(Compound (I), X=4-Cl, n=1, R¹=H, R²=H, R³=Me, R⁴=Me, Isomer Form C) and Compound No. I-151(Compound (I), X=4-Cl, n=1, R¹=Me, R²=Me, R³=H, R⁴=H, Isomer Form C))

Under nitrogen flow, 60% sodium hydride (45 mg, 1.13 mmol) was washed with hexane, and then suspended in anhydrous DMF (2 ml), and while cooling with ice 1H-1,2,4-triazole (78 mg, 1.13 mmol) was added. After stirring at room temperature for 5 minutes, an anhydrous DMF (1 ml) solution of 9-(4-chlorobenzyl)-5,5-dimethyl-1-oxadispiro[2.0.2.3]nonane (Mixture of Compound (II), X=4-Cl, n=1, R¹=H, R²=H, R³=Me, R⁴=Me and Compound (II), X=4-Cl, n=1, R¹=Me, R²=Me, R³=H, R⁴=H) (314 mg, 1.13 mmol) was added, and stirring was continued for 5 hours at 90 degrees C. The reaction solution was poured into an iced water, and extracted with ethyl acetate. The organic layer was washed with water, saturated brine, and dried over anhydrous sodium sulfate. The solvent was distilled off under reduced pressure, and the resultant crude product was purified by silica gel column chromatography (eluent, hexane-ethyl acetate, 1:1) to obtain the title compound.

Compound No. I-101

Product: 18 mg, Yield: 5%, Tan oil.

¹H-NMR (CDCl₃) delta 0.26 (d, J=4.8 Hz, 1H), 0.79 (d, J=4.8 Hz, 1H), 1.17 (s, 3H), 1.3-1.5 (m, 2H), 1.3-1.5 (m, 2H), 1.38 (s, 3H), 1.5-1.6 (m, 1H), 1.9-2.1 (m, 3H), 2.19 (dd, J=14.0, 11.8 Hz, 1H), 2.81 (s, 1H), 4.24 (s, 2H), 6.92 (d, J=8.4 Hz, 2H), 7.18 (d, J=8.4 Hz, 2H), 7.99 (s, 1H), 8.20 (s, 1H).

Compound No. I-151

Product: 17 mg, Yield: 4%, Tan oil.

¹H-NMR (CDCl₃) delta 0.18 (d, J=4.0 Hz, 1H), 1.04 (d, J=4.0 Hz, 1H), 1.16 (s, 3H), 1.33 (s, 3H), 1.4-1.5 (m, 2H), 1.5-1.6 (m, 1H), 1.7-1.8 (m, 1H), 1.9-2.0 (m, 1H), 2.29 (dd, J=13.7, 11.0 Hz, 1H), 2.57 (dd, J=13.7, 4.6 Hz, 1H), 3.37 (s, 1H), 4.23 (d, J=14.2 Hz, 1H), 4.34 (d, J=14.2 Hz, 1H), 6.84 (d, J=8.4 Hz, 2H), 7.16 (d, J=8.4 Hz, 2H), 7.99 (s, 1H), 8.13 (s, 1H).

By the methods analogous to Production Example 1 to 5 described above, the following Compounds (I) were synthesized. The characteristics of each compound are shown in Table 21 to Table 23.

TABLE 21

| Compound No | Description | ¹H-NMR(CDCl₃) 400 MHz, δ |
|---|---|---|
| I-5 | Amber oil | 0.33-0.36 (m, 1H), 0.47-0.50 (m, 1H), 0.57-0.61 (m, 1H), |
| | | 0.74-0.76 (m, 1H), 1.61-1.63 (m, 2H), 1.73-1.76 (m, 2H), |
| | | 2.15-2.17 (m, 1H), 2.60 (dd, J = 13.5, 10.8 Hz, 1H), 2.74 (dd, |
| | | J = 13.5, 4.3 Hz, 1H), 3.40 (bs, 1H), 4.08 (d, J = 14.0 Hz, 1H), |
| | | 4.25 (d, J = 14.0 Hz, 1H), 7.12-7.14 (m, 3H), 7.28 (d, J = 7.8 Hz, |
| | | 1H), 7.96 (s, 1H), 8.14 (s, 1H). |
| I-6 | Brown solid, | −0.19-−0.16 (m, 1H), 0.12-0.14 (m, 1H), 0.28-0.30 (m, 1H), |
| | Melting point | 0.59-0.62 (m, 1H), 1.47-1.53 (m, 2H), 1.89-1.98 (m, 2H), |
| | 102.5-103.8° C. | 2.41-2.47 (m, 1H), 2.58 (dd, J = 12.9, 12.0 Hz, 1H), 3.12 (dd, |
| | | J = 12.9, 3.2 Hz, 1H), 3.28 (bs, 1H), 4.26 (d, J = 14.1 Hz, 1H), |
| | | 4.39 (d, J = 14.1 Hz, 1H), 7.12-7.23 (m, 3H), 7.34 (d, J = 7.9 Hz, |
| | | 1H), 7.97 (s, 1H), 8.20 (s, 1H). |
| I-21 | White solid, | 0.34 (ddd, J = 9.2, 6.0, 4.1 Hz, 1H), 0.5-0.6 (m, 2H), 0.83 (ddd, J = 9.7, |
| | Melting point | 6.0, 4.1 Hz, 1H), 1.5-1.6 (m, 2H), 1.6-1.7 (m, 1H), 1.7-1.8 (m, |
| | 85-86° C. | 1H), 2.0-2.1 (m, 1H), 2.4-2.5 (m, 2H), 3.19 (s, 1H), 4.07 (d, J = 14.0 Hz, |
| | | 1H), 4.20 (d, J = 14.0 Hz, 1H), 7.13 (d, J = 8.0 Hz, 2H), |
| | | 7.48 (d, J = 8.0 Hz, 2H), 7.97 (s, 1H), 8.16 (s, 1H). |
| I-22 | White solid, | −0.30 (ddd, J = 10.2, 5.9, 5.1 Hz, 1H), 0.09 (ddd, J = 9.5, 5.9, |
| | Melting point | 4.0 Hz, 1H), 0.25 (ddd, J = 9.5, 5.9, 5.1 Hz, 1H), 0.61 (ddd, J = 10.2, |
| | 135.5-135.6° C. | 5.9, 4.0 Hz, 1H), 1.4-1.5 (m, 2H), 1.8-2.0 (m, 2H), |
| | | 2.3-2.4 (m, 2H), 3.17 (d, J = 10.2 Hz, 1H), 3.35 (s, 1H), 4.26 (d, J = 14.0 Hz, |
| | | 1H), 4.34 (d, J = 14.0 Hz, 1H), 7.29 (d, J = 8.2 Hz, 2H), |
| | | 7.54 (d, J = 8.1 Hz, 1H), 7.99 (s, 1H), 8.16 (s, 1H). |
| I-27 | Amber oil | 0.32-0.35 (m, 1H), 0.45-0.48 (m, 1H), 0.51-0.57 (m, 1H), |
| | | 0.74-0.78 (m, 1H), 1.51-1.56 (m, 2H), 1.64-1.72 (m, 2H), |
| | | 2.29 (s, 3H), 2.31-2.42 (m, 3H), 3.04 (bs, 1H), 4.05 (d, |
| | | J = 14.1 Hz, 1H), 4.19 (d, J = 14.1 Hz, 1H), 6.91 (d, J = 7.6 Hz, |
| | | 2H), 7.04 (d, J = 7.6 Hz, 2H), 7.97 (s, 1H), 8.14 (s, 1H). |
| I-28 | White solid, | −0.33-−0.29 (m, 1H), 0.05-0.09 (m, 1H), 0.21-0.24 (m, 1H), |
| | Melting point | 0.55-0.59 (m, 1H), 1.40-1.44 (m, 2H), 1.92-1.98 (m, 2H), |
| | 134.2-135.5° C. | 2.32 (s, 3H), 2.30-2.32 (m, 2H), 3.03-3.06 (m, 1H), 3.19 (bs, |
| | | 1H), 4.26 (d, J = 14.0 Hz, 1H), 4.34 (d, J = 14.0 Hz, 1H), |
| | | 7.07-7.09 (m, 4H), 7.97 (s, 1H), 8.13 (s, 1H). |

TABLE 22

| | | |
|---|---|---|
| I-49 | White solid, | 0.35 (ddd, J = 9.5, 6.0, 4.1 Hz, 1H), 0.50 (ddd, J = 9.5, 6.0, 4.8 Hz, |
| | Melting point | 1H), 0.57 (ddd, J = 10.0, 6.0, 4.8 Hz, 1H), 0.81 (ddd, J = 10.0, 6.0, |
| | 137-138° C. | 4.1 Hz, 1H), 1.5-1.7 (m, 2H), 1.7-1.8 (m, 1H), 2.0-2.1 (m, 1H), |
| | | 2.42 (dd, J = 13.5, 10.5 Hz, 1H), 2.50 (dd, J = 13.5, 4.8 Hz, 1H), |
| | | 3.12 (s, 1H), 4.07 (d, J = 14.0 Hz, 1H), 4.22 (d, J = 14.0 Hz, 1H), |
| | | 7.09 (d, J = 8.3 Hz, 2H), 7.32 (ddt, J = 8.1, 6.7, 1.3 Hz, 1H), |
| | | 7.42 (t, J = 8.1 Hz, 2H), 7.47 (d, J = 8.3 Hz, 2H), 7.56 (dd, J = 8.1, |
| | | 1.3 Hz, 2H), 7.98 (s, 1H), 8.17 (s, 1H). |
| I-50 | White solid, | −0.30 (ddd, J = 10.2, 5.9, 5.1 Hz, 1H), 0.09 (ddd, J = 9.5, 5.9, |
| | Melting point | 4.0 Hz, 1H), 0.25 (ddd, J = 9.5, 5.9, 5.1 Hz, 1H), 0.61 (ddd, J = 10.2, |
| | 131-132° C. | 5.9, 4.0 Hz, 1H), 1.4-1.5 (m, 2H), 1.9-2.0 (m, 2H), |
| | | 2.4-2.5 (m, 2H), 3.14 (dd, J = 19.9, 10.2 Hz, 1H), 3.29 (s, 1H), 4.28 (d, J = 14.0 Hz, |
| | | 1H), 4.37 (d, J = 14.0 Hz, 1H), 7.25 (d, J = 8.3 Hz, 2H), |
| | | 7.33 (ddt, J = 8.1, 6.7, 1.3 Hz, 1H), 7.43 (t, J = 8.1 Hz, 2H), |
| | | 7.52 (d, J = 8.3 Hz, 2H), 7.58 (dd, J = 8.1, 1.3 Hz, 2H), 7.99 (s, 1H), |
| | | 8.15 (s, 1H). |
| I-61 | Yellow | 0.30-0.35 (m, 1H), 0.47-0.51 (m, 1H), 0.54-0.58 (m, 1H), |
| | syrup-like | 0.75-0.79 (m, 1H), 1.58-1.62 (m, 2H), 1.73-1.76 (m, 2H), |
| | substance | 2.06-2.12 (m, 1H), 2.56 (dd, J = 13.5, 10.8 Hz, 1H), 2.68 (dd, |
| | | J = 13.5, 4.5 Hz, 1H), 3.46 (bs, 1H), 4.07 (d, J = 14.1 Hz, 1H), |
| | | 4.23 (d, J = 14.1 Hz, 1H), 7.03-7.15 (m, 2H), 7.30 (d, J = 1.9 Hz, |
| | | 1H), 7.96 (s, 1H), 8.14 (s, 1H). |
| I-62 | Yellow | −0.21-−0.18 (m, 1H), 0.13-0.16 (m, 1H), 0.29-0.32 (m, 1H), |
| | syrup-like | 0.58-0.61 (m, 1H), 1.48-1.53 (m, 2H), 1.88-1.98 (m, 2H), |
| | substance | 2.35-2.38 (m, 1H), 2.55 (dd-like, J = 13.0, 12.0 Hz, 1H), |
| | | 3.08 (dd, J = 13.0, 3.2 Hz, 1H), 3.20 (bs, 1H), 4.24 (d, |
| | | J = 14.1 Hz, 1H), 4.37 (d, J = 14.1 Hz, 1H), 7.16-7.17 (m, 2H), |
| | | 7.37 (d, J = 1.9 Hz, 1H), 7.98 (s, 1H), 8.18 (s, 1H). |
| I-63 | Yellow | 0.33-0.36 (m, 1H), 0.49-0.55 (m, 2H), 0.79-0.83 (m, 1H), |
| | syrup-like | 1.50-1.54 (m, 2H), 1.74-1.78 (m, 2H), 2.00-2.03 (m, 1H), |
| | substance | 2.29-2.42 (m, 2H), 3.20 (bs, 1H), 4.06 (d, J = 14.1 Hz, 1H), |
| | | 4.19 (d, J = 14.1 Hz, 1H), 6.85 (d, J = 8.1 Hz, 1H), 7.13-7.14 (m, |
| | | 1H), 7.29 (d, J = 8.1 Hz, 1H), 7.98 (s, 1H), 8.16 (s, 1H). |
| I-64 | White solid, | −0.31-−0.29 (m, 1H), 0.09-0.12 (m, 1H), 0.23-0.26 (m, 1H), |
| | Melting point | 0.59-0.62 (m, 1H), 1.40-1.48 (m, 2H), 1.89-1.97 (m, 2H), |
| | 125.8-127.3° C. | 2.27-2.29 (m, 2H), 3.06-3.08 (m, 1H), 3.32 (bs, 1H), 4.23 (d, |
| | | J = 14.0 Hz, 1H), 4.31 (d, J = 14.0 Hz, 1H), 7.02 (d, J = 8.2 Hz, |
| | | 1H), 7.27-7.28 (m, 1H), 7.35 (d, J = 8.2 Hz, 1H), 7.98 (s, 1H), |
| | | 8.15 (s, 1H). |

TABLE 23

| | | |
|---|---|---|
| I-301 | White solid, | 0.3-0.4 (m, 1H), 0.5-0.6 (m, 1H), 0.6-0.8 (m, 2H), 1.4-1.6 (m, 2H), |
| | Melting point | 1.6-1.8 (m, 2H), 1.82 (bs, 1H), 2.02 (ddd, J = 14.9, 8.1, 7.0 Hz, 1H), |
| | 113-114° C. | 2.32 (bd, J = 7.5 Hz, 2H), 3.79 (d, J = 14.5 Hz, 1H), 3.87 (d, J = 14.5 Hz, |
| | | 1H), 6.95 (d, J = 8.4 Hz, 2H), 7.05 (bs, 1H), 7.07 (bs, 1H), |
| | | 7.20 (d, J = 8.4 Hz, 2H), 7.56 (s, 1H). |
| I-302 | Tan solid | −0.03 (ddd, J = 10.2, 5.9, 5.1 Hz, 1H), 0.13 (ddd, J = 9.2, 5.9, |
| | Melting point | 4.3 Hz, 1H), 0.3-0.4 (m, 1H), 0.4-0.5 (m, 1H), 1.3-1.5 (m, 2H), |
| | 166-167° C. | 1.84 (bs, 1H), 1.8-2.0 (m, 2H), 2.2-2.3 (m, 1H), 2.38 (dd, J = 13.0, |
| | | 11.8 Hz, 1H), 3.00 (dd, J = 13.0, 3.5 Hz, 1H), 3.96 (d, J = 14.3 Hz, |
| | | 1H), 4.08 (d, J = 14.3 Hz, 1H), 7.02 (bs, 1H), 7.03 (bs, 1H), 7.08 (d, |
| | | J = 8.4 Hz, 2H), 7.25 (d, J = 8.4 Hz, 2H), 7.54 (s, 1H). |

The Compounds (IV) used as described above can be synthesized by the following Production Example 6 to 9 and analogous methods.

### Production Example 6

### Synthesis of 5-(4-chlorobenzyl)-4-spiro[2.4]heptanone (Compound (IV), X=4-Cl, n=1, R¹=H, R²=H, R³=H, R⁴=H)

### (1) Synthesis of Intermediate, Methyl 1-(2-bromoethyl)-3-(4-chlorobenzyl)-2-oxocyclopentanecarboxylaLe (Compound (VI), X=4-Cl, n=1, R¹=H, R²=H, R³=H, R⁴=H, R⁵=Me, Z¹=Br)

Under nitrogen flow, 60% sodium hydride (0.83 g, 20.7 mmol) was washed with hexane, and then suspended in anhydrous DMF (5 ml) solution, while cooling with ice, an anhydrous DMF (10 ml) solution of methyl 3-(4-chlorobenzyl)-2-oxocyclopentanecarboxylate (Compound (VII), X=4-Cl, n=1, R¹=H, R²=H, R³=H, R⁴=H, R⁵=Me) (5.02 g, 18.8 mmol) was added dropwise over 10 minutes. After stirring at room temperature for 5 minutes, 1,2-dibromoethane (Compound (VIII), R¹=H, R²=H, R³=H, R⁴=H, Z¹=Br, Z²=Br) (3.97 g, 20.7 mmol) was added, and stirred at 90 degrees C. for 2.5 hours. The reaction solution was poured into an iced water, and extracted with ethyl acetate. The organic layer was washed with water, saturated brine, and dried over anhydrous sodium sulfate. The solvent was distilled off under reduced pressure, the crude title compound was obtained.

Crude product: 5.48 g, crude yield: 78%, Pale yellow oil.

### (2) Synthesis of Intermediate, 2-(2-bromoethyl)-5-(4-chlorobenzyl)cyclopentanone (Compound (V), X=4-Cl, n=1, R¹=H, R²=H, R³=H, R⁴=H, Z¹=Br)

The crude methyl 1-(2-bromoethyl)-3-(4-chlorobenzyl)-2-oxocyclopentanecarboxylate obtained above (Compound (VI), X=4-Cl, n=1, R¹=H, R²=H, R³=H, R⁴=H, R⁵=Me, Z¹=Br) (5.48 g, 14.7 mmol) was dissolved in acetic acid (5 ml), combined with 48% hydrobromic acid (4.94 g, 29.3 mmol), and heated under reflux for 3 hours. The reaction solution was poured into an iced water, and then neutralized with a 10% aqueous solution of sodium hydroxide, and extracted with ethyl acetate. The organic layer was washed with water, saturated brine, and dried over anhydrous sodium sulfate. The solvent was distilled off under reduced pressure, and a crude title compound was obtained.

Crude product: 4.61 g, crude yield: 99%, Yellow oil.

¹H-NMR (CDCl₃) delta 1.3-1.5 (m, 2H), 1.6-1.8 (m, 1H), 1.8-1.9 (m, 1H), 2.0-2.1 (m, 1H), 2.2-2.3 (m, 2H), 2.3-2.4 (m, 1H), 2.61 (dd, J=14.2, 9.0 Hz, 1H), 3.10 (dd, J=13.9, 4.4 Hz, 1H), 3.4-3.5 (m, 1H), 3.5-3.6 (m, 1H), 7.08 (d, J=8.4 Hz, 2H), 7.24 (d, J=8.4 Hz, 2H).

### (3) Synthesis of 5-(4-chlorobenzyl)-4-spiro[2.4]heptanone (Compound (IV), X=4-Cl, n=1, R¹=H, R²=H, R³=H, R⁴=H)

Under nitrogen flow, 60% sodium hydride (1.75 g, 43.8 mmol) was washed with hexane, and then suspended in anhydrous THF (15 ml), and then while heating under reflux an anhydrous THF (5 ml) solution of the crude product of 2-(2-bromoethyl)-5-(4-chlorobenzyl)cyclopentanone (Compound (V), X=4-Cl, n=1, R¹=H, R²=H, R³=H, R⁴=H, Z¹=Br) (4.61 g, 14.6 mmol) was added dropwise over 10 minutes, and then heated under reflux for 6 hours. The reaction solution was poured into an iced water, and extracted with ethyl acetate. The organic layer was washed with a saturated brine, and dried over anhydrous sodium sulfate. The solvent was distilled off under reduced pressure, and the resultant crude product was purified by a silica gel column chromatography (eluent, hexane-ethyl acetate, 19:1) to obtain the title compound.

Product: 1.93 g, Yield: 56% Yield from methyl (3-(4-chlorobenzyl)-2-oxocyclopentanecarboxylate (Compound (VII), X=4-Cl, n=1, R¹=H, R²=H, R³=H, R⁴=H, R⁵=Me) 44%), Pale yellow oil.

¹H-NMR (CDCl₃) delta 0.8-1.0 (m, 2H), 1.0-1.1 (m, 1H), 1.2-1.3 (m, 1H), 1.6-1.8 (m, 2H), 2.0-2.2 (m, 2H), 2.5-2.6 (m, 2H), 3.13 (d, J=9.7 Hz, 1H), 7.11 (d, J=8.3 Hz, 2H), 7.24 (d, J=8.3 Hz, 2H).

### Production Example 7

### Synthesis of 5-(4-chlorobenzyl)-1,1-dimethyl-4-spiro[2.4]heptanone (Mixture of Compound (IV), X=4-Cl, n=1, R¹=Me, R²=Me, R³=H, R⁴=H and Compound (IV), X=4-Cl, n=1, R¹=H, R²=H, R³=Me, R⁴=Me)

### (1) Synthesis of Intermediate, 2-(4-chlorobenzyl)-5-isopropylidenecyclopentanone (Compound (IX), X=4-Cl, n=1, R⁶=Me, R⁷=Me)

2-(4-Chlorobenzyl)cyclopentanone (Compound (X), X=4-Cl, n=1) (5.10 g, 24.4 mmol), acetone (7.16 g, 123.3 mmol) were dissolved in methanol (5 ml) combined with potassium hydroxide (1.37 g, 24.4 mmol), and heated under reflux for 2 hours. The reaction solution was poured into an iced water, and extracted with ethyl acetate. The organic layer was washed with water, saturated brine, and dried over anhydrous sodium sulfate. The solvent was distilled off under reduced pressure, and the resultant crude product was purified by a silica gel column chromatography (eluent, hexane-ethyl acetate 19:1) to obtain the title compound.

Crude product: 4.27 g, Yield: 70, Pale yellow solid.

¹H-NMR (CDCl₃) delta 1.4-1.5 (m, 1H), 1.83 (s, 3H), 1.9-2.0 (m, 1H), 2.25 (s, 3H), 2.4-2.6 (m, 3H), 2.51 (d, J=9.4 Hz, 1H), 3.17 (d, J=9.4 Hz, 1H), 7.11 (d, J=8.4 Hz, 2H), 7.24 (d, J=8.4 Hz, 2H).

### (2) Synthesis of 5-(4-chlorobenzyl)-1,1-dimethyl-4-spiro[2.4]heptanone (Mixture of Compound (IV), X=4-Cl, n=1, R¹=Me, R²=Me, R³=H, R⁴=H and Compound (IV), X=4-Cl, n=1, R¹=H, R²=H, R³=Me, R⁴=Me)

Under nitrogen flow, 60% sodium hydride (345 mg, 8.6 mmol) was washed with hexane, and then suspended in DMSO (4 ml), and then trimethylsulfoxonium bromide (1.49 g, 8.6 mmol) was added. After stirring at room temperature for 5 minutes, a DMSO (3 ml) solution of a crude 2-(4-chlorobenzyl)-5-isopropylidenecyclopentanone (Compound (IX), X=4-Cl, n=1, R⁶=Me, R⁷=Me) (2.15 g, 8.6 mmol) obtained above was added, and stirring was continued at room temperature for 6 hours. The reaction solution was poured into an iced water, and extracted with ethyl acetate. The organic layer was washed with water, saturated brine, and dried over anhydrous sodium sulfate. The solvent was distilled off under reduced pressure, and the resultant crude product was purified by a silica gel column chromatography (eluent, hexane-ethyl acetate 19:1) to obtain the title compound.

Product: 2.18 g, Yield: 96%, White solid.

¹H-NMR (CDCl₃) delta 0.77 (d, J=3.5 Hz, 1H), 1.13 (s, 3H), 1.24 (s, 3H), 1.29 (d, J=3.5 Hz, 1H), 1.5-1.6 (m, 1H), 1.6-1.7 (m, 1H), 2.0-2.2 (m, 2H), 2.5-2.6 (m, 2H), 3.15 (d, J=9.9 Hz, 1H), 7.11 (d, J=8.4 Hz, 2H), 7.24 (d, J=8.4 Hz, 2H).

### Production Example 8

### Synthesis of 5-(4-fluorobenzyl)-4-spiro[2.4]heptanone (Compound (IV), X=4-F, n=1, R¹=Me, R²=Me, R³=H, R⁴=H)

### (1) Synthesis of Intermediate, 4-oxaspiro[2.4]-5-heptanecarboxylic acid methyl ester (Compound (XIV), X=4-F, n=1, R¹=H, R²=H, R³=H, R⁴=H, R⁸=Me)

Under nitrogen flow, 60% sodium hydride (1.54 g, 38.7 mmol) was washed with hexane, and then suspended in dimethyl carbonate (18 ml), and then 10 drops of dehydrated methanol was added. While heating under reflux, a dimethyl carbonate (Compound (XVI), R⁸=Me, Y=OMe) (8 ml) solution of spiro[2.4]-4-heptanone (Compound (XV), R¹=H, R²=H, R³=H, R⁴=H)(2.84 g, 25.8 mmol) was added dropwise over 10 minutes (total amount of dimethyl carbonate used was 23.72 g, 258 mmol), and then heating under reflux was continued for 3.5 hours. The reaction solution was poured into an iced water, and extracted with ethyl acetate. The organic layer was washed with water, saturated brine, and dried over anhydrous sodium sulfate. The solvent was distilled off under reduced pressure, the resultant crude product was purified by a silica gel column chromatography (eluent, hexane-ethyl acetate=9:1) to obtain the crude title compound.

Product: 3.21 g, Yield: 74%, Tan oil.

¹H-NMR (CDCl₃) delta 0.97 (dd, J=8.9, 5.1 Hz, 2H), 1.26 (ddd, J=15.4, 9.9, 3.8 Hz, 2H), 2.02 (ddd, J=12.7, 7.6, 4.1 Hz, 1H), 2.11 (ddd, J=12.7, 9.2, 7.0 Hz, 1H), 2.3-2.4 (m, 1H), 2.4-2.5 (m, 1H), 3.39 (t, J=9.2 Hz, 1H), 3.76 (s, 3H).

### (2) Synthesis of methyl 5-(4-fluorobenzyl)-4-oxaspiro[2.4]heptane-5-carboxylate (Compound (XII), X=4-F, n=1, R¹=H, R²=H, R³=H, R⁴=H, R⁸=Me)

Under nitrogen flow, 60% sodium hydride (270 mg, 6.8 mmol) was washed with hexane, and then suspended in anhydrous DMF (3 ml), and then while cooling with ice an anhydrous DMF (3 ml) solution of 4-oxaspiro[2.4]heptane-5-carboxylic acid methyl ester (Compound (XIV), X=4-F, n=1, R¹=H, R²=H, R³=H, R⁴=H, R⁸=Me) (758 mg, 4.5 mmol) was added dropwise over 5 minutes. After stirring at 0 degrees C. for 5 minutes, 4-fluorobenzyl bromide (665 mg, 4.5 mmol) was added, and stirring was continued for 3 hours at 80 degrees C. The reaction solution was poured into an iced water, and extracted with ethyl acetate. The organic layer was washed with water, saturated brine, and dried over anhydrous sodium sulfate. The solvent was distilled off under reduced pressure, and a crude title compound was obtained.

Crude product: 1.18 g, crude yield: 94%, Tan oil.

¹H-NMR (CDCl₃) delta 0.78 (ddd, J=9.1, 7.2, 3.2 Hz, 1H), 0.95 (ddd, J=9.1, 7.2, 3.5 Hz, 1H), 1.09 (ddd, J=9.9, 7.2, 3.2 Hz, 1H), 1.30 (ddd, J=9.9, 7.2, 3.5 Hz, 1H), 1.61 (ddd, J=12.6, 7.5, 4.1 Hz, 1H), 2.00 (ddd, J=12.6, 8.6, 7.5 Hz, 1H), 2.1-2.2 (m, 1H), 2.49 (ddd, J=12.7, 7.2, 4.1 Hz, 1H), 3.09 (d, J=14.0 Hz, 1H), 3.23 (d, J=14.0 Hz, 1H), 3.73 (s, 3H), 6.93 (d, J=8.4 Hz, 2H), 7.09 (d, J=8.4 Hz, 2H).

### (3) Synthesis of 5-(4-fluorobenzyl)-4-spiro[2.4]heptanone (Compound (IV), X=4-F, n=1, R¹=H, R²=H, R³=H, R⁴=H)

The crude methyl 5-(4-fluorobenzyl)-4-oxaspiro[2.4]heptane-5-carboxylate obtained above (Compound (XII), X=4-F, n=1, R¹=H, R²=H, R³=H, R⁴=H, R⁸=Me) (1.18 g, 4.3 mmol) was dissolved in 2-propanol (3 ml), and a solution of sodium hydroxide (269 mg, 6.4 mmol) in water (1 ml) was added, and stirring was continued at 60 degrees C. for 5 hours. The reaction solution was poured into an iced water, and extracted with ethyl acetate. The organic layer was washed with water, saturated brine, and dried over anhydrous sodium sulfate. The solvent was distilled off under reduced pressure, the resultant crude product was purified by a silica gel column chromatography (eluent, hexane-ethyl acetate=9:1) to obtain the title compound.

Product: 649 mg, Yield: 70%, Pale yellow oil.

¹H-NMR (CDCl₃) delta 0.82 (ddd, J=9.1, 6.8, 2.7 Hz, 1H), 0.90 (ddd, J=9.1, 6.8, 3.3 Hz, 1H), 1.09 (ddd, J=9.9, 6.8, 2.7 Hz, 1H), 1.25 (ddd, J=9.9, 6.8, 3.3 Hz, 1H), 1.6-1.8 (m, 2H), 2.0-2.2 (m, 2H), 2.5-2.6 (m, 1H), 2.58 (d, J=9.7 Hz, 1H), 3.14 (d, J=9.7 Hz, 1H), 6.95 (d, J=8.4 Hz, 2H), 7.12 (d, J=8.4 Hz, 2H).

### Production Example 9

### Synthesis of 5-(4-chlorobenzyl)-4-spiro[2.4]heptanone (Compound (IV), X=4-Cl, n=1, R¹=H, R²=H, R³=H, R⁴=H)

### (1) Synthesis of Intermediate, Methyl 1-(2-methoxyethyl)-3-(4-chlorobenzyl)-2-oxocyclopentanecarboxylate (Compound (XVIII), X=4-Cl, n=1, R¹=H, R²=H, R³=H, R⁴=H, R⁵=Me, R⁹=Me)

Under nitrogen flow, 60% sodium hydride (1.64 g, 41.0 mmol) was washed with hexane, and then suspended in anhydrous DMF (2 ml) solution, and then while cooling with ice an anhydrous DMF (10 ml) solution (dissolved with heating) of methyl 3-(4-chlorobenzyl)-2-oxocyclopentanecarboxylate (Compound (VII), X=4-Cl, n=1, R¹=H, R²=H, R³=H, R⁴=H, R⁵=Me) (10 g, 0.37 mmol) was added dropwise over 30 minutes. The DMF solution was washed with anhydrous DMF (3 ml), and added dropwise to the reaction solution. At room temperature, stirring was continued for about 1 hour, bromoethyl methyl ether (Compound (XVII), R¹=H, R²=H, R³=H, R⁴=H, Z⁴=Br, R⁹=Me) (6.25 g, 45.0 mmol) was added, and stirring was continued at 70 degrees C. for 7 hours. The reaction solution was poured into an iced water, made acidic with a dilute hydrochloric acid, and extracted with ethyl acetate. The organic layer was washed with a saturated brine, and the solvent was distilled off under reduced pressure.

The resultant crude product was purified by a silica gel column chromatography (eluent, hexane-ethyl acetate=10:1-7:1) to obtain the title compound.

Product: 9.34 g, Yield: 77%, Colorless oil.

¹H-NMR (CDCl₃) delta 1.5-1.9 (m, 2H), 1.9-2.7 (m, 6H), 2.8-3.8 (m, 6H), 3.61 (m, 1.5H), 3.69 (m, 1.5H), 7.0-7.2 (m, 2H), 7.2-7.3 (m, 2H).

### (2) Synthesis of Intermediate, 2-(2-bromoethyl)-5-(4-chlorobenzyl)cyclopentanone (Compound (Va), X=4-Cl, n=1, R¹=H, R²=H, R¹=H, R⁴=H, Z⁵=Br)

Methyl 1-(2-methoxyethyl)-3-(4-chlorobenzyl)-2-oxocyclopentanecarboxylate obtained above (Compound (XVIII), X=4-Cl, n=1, R¹=H, R²=H, R³=H, R⁴=H, R⁵=Me, R⁹=Me) (1.02 g, 3.13 mmol) was combined with acetic acid (0.5 ml), and then with 48% hydrobromic acid (2 ml, 17.3 mmol), and then stirred while heating at 120 degrees C. for 15 hours. After pouring the reaction solution into an iced water, extraction was made with chloroform. The organic layer was washed with a saturated brine, and dried over anhydrous sodium sulfate. The resultant crude product was purified by a silica gel column chromatography (eluent, hexane-ethyl acetate=5:1) to obtain the title compound.

Product: 0.63 g, Yield: 64%, Oil.

### (3) Synthesis of 5-(4-chlorobenzyl)-4-spiro[2.4]heptanone (Compound (IV), X=4-Cl, n=1, R¹=H, R²=H, R³=H, R⁴=H)

2-(2-Bromoethyl)-5-(4-chlorobenzyl)cyclopentanone obtained above (Compound (V), X=4-Cl, n=1, R¹=H, R²=H, R³=H, R⁴=H, Z⁵=Br) (0.64 g, 2.03 mmol) was dissolved in ethanol (1 ml), combined with potassium carbonate (0.42 g, 3.04 mmol), and stirred at about 70 degrees C. for 2 hours. After filtering the reaction mixture, the filtrate was concentrated to obtain a crude product. The resultant crude product was purified by a silica gel column chromatography (eluent, hexane-ethyl acetate=10:1) to obtain the title compound.

Product: 0.36 g, Yield: 76%, Colorless oil.

The following Compounds (IV) were synthesized by the methods analogous to the abovementioned Production Examples 6 to 9. The structures of respective compounds are listed in Table 24. The Characteristics of the respective compounds are listed in Table 24.

TABLE 24

| Compound No | R¹ | R² | R³ | R⁴ | Xn |
|---|---|---|---|---|---|
| IV-5 | H | H | H | H | 4-Me |
| IV-6 | H | H | H | H | 2-Cl |
| IV-7 | H | H | H | H | 3-Cl |
| IV-8 | H | H | H | H | 4-CF₃ |
| IV-9 | H | H | H | H | 4-Ph |
| IV-10 | H | H | H | H | 2,4-Cl₂ |
| IV-11 | H | H | H | H | 3,4-Cl₂ |

TABLE 25

| Compound No | Description | ¹H-NMR(CDCl₃) 400 MHz, δ |
|---|---|---|
| IV-5 | Brown oil | 0.85-0.88 (m, 2H), 1.10-1.14 (m, 1H), 1.25-1.28 (m, 1H), |
| | | 1.70-1.75 (m, 2H), 2.05-2.12 (m, 2H), 2.32 (s, 3H), |
| | | 2.52-2.55 (m, 2H), 3.15 (d-like, J = 9.9 Hz, 1H), 7.09 (s-like, |
| | | 4H). |
| IV-6 | Brown oil | 0.88-0.91 (m, 2H), 1.14-1.17 (m, 1H), 1.24-1.27 (m, 1H), |
| | | 1.70-1.78 (m, 2H), 2.05-2.09 (m, 2H), 2.64-2.70 (m, 2H), |
| | | 3.37 (dd, J = 8.6, 3.2 Hz, 1H), 7.16-7.19 (m, 2H), 7.23 (dd, |
| | | J = 7.0, 2.2 Hz, 1H), 7.35 (d, J = 7.2 Hz, 1H). |
| IV-7 | Yellow oil | 0.84 (ddd, J = 9.1, 6.7, 2.8 Hz, 1H), 0.91 (ddd, J = 9.1, 6.7, 3.3 Hz, |
| | | 1H), 1.11 (ddd, J = 10.0 6.7, 2.8 Hz, 1H), 1.26 (ddd, J = 10.0, 6.7, |
| | | 3.3 Hz, 1H), 1.6-1.8 (m, 2H), 2.0-2.2 (m, 2H), 2.5-2.6 (m, 1H), |
| | | 2.55 (d, J = 10.0 Hz, 1H), 3.17 (d, J = 10.0 Hz, 1H), 7.06 (dt, J = 6.8, |
| | | 1.8 Hz, 1H), 7.1-7.3 (m, 3H). |
| IV-8 | Colorless oil | 0.85 (ddd, J = 9.1, 6.7, 2.9 Hz, 1H), 0.91 (ddd, J = 9.1, 6.7, 3.5 Hz, |
| | | 1H), 1.11 (ddd, J = 9.9, 6.7, 2.9 Hz, 1H), 1.27 (ddd, J = 9.9, 6.7, |
| | | 3.5 Hz, 1H), 1.7-1.8 (m, 1H), 1.8-1.9 (m, 1H), 2.0-2.2 (m, 2H), |
| | | 2.5-2.7 (m, 2H), 3.24 (dd, J = 12.9, 3.5 Hz, 1H), 7.30 (d, J = 8.1 Hz, |
| | | 2H), 7.54 (d, J = 8.1 Hz, 2H). |
| IV-9 | White solid, | 0.85 (ddd, J = 9.1, 6.7, 2.9 Hz, 1H), 0.90 (ddd, J = 9.1, 6.7, 3.3 Hz, |
| | Melting point | 1H), 1.13 (ddd, J = 9.9, 6.7, 2.9 Hz, 1H), 1.27 (ddd, J = 9.9, 6.7, |
| | 104.0-105.0° C. | 3.3 Hz, 1H), 1.7-1.8 (m, 2H), 2.0-2.1 (m, 1H), 2.1-2.2 (m, 1H), |
| | | 2.61 (dd, J = 18.4, 9.1 Hz, 1H), 2.6-2.7 (m, 1H), 3.24 (dd, J = 18.4, |
| | | 9.1 Hz, 1H), 7.26 (d, J = 8.3 Hz, 2H), 7.33 (ddt, J = 8.0, 6.7, 1.3 Hz, |
| | | 1H), 7.43 (t, J = 8.0 Hz, 2H), 7.52 (d, J = 8.3 Hz, 2H), 7.58 (dd, J = 8.3, |
| | | 1.3 Hz, 2H). |
| IV-10 | Yellow oil | 0.86-0.91 (m, 2H), 1.13-1.16 (m, 1H), 1.26-1.28 (m, 1H), |
| | | 1.60-1.78 (m, 2H), 2.05-2.10 (m, 2H), 2.66-2.68 (m, 2H), |
| | | 3.30 (dd-like, J = 11.1, 8.9 Hz, 1H), 7.17 (s-like, 2H), |
| | | 7.37 (s-like, 1H). |
| IV-11 | Brown oil | 0.85-0.93 (m, 2H), 1.10-1.13 (m, 1H), 1.21-1.27 (m, 1H), |
| | | 1.64-1.75 (m, 2H), 2.05-2.12 (m, 2H), 2.54-2.58 (m, 2H), |
| | | 3.11-3.13 (m, 1H), 7.02 (d, J = 8.2 Hz, 1H), 7.27 (s-like, 1H), |
| | | 7.34 (d, J = 8.2 Hz, 1H). |

The following Compounds (XII) were synthesized by the methods analogous to the abovementioned Production Example 8. The structures of respective compounds are listed in Table 26. The Characteristics of the respective compounds are listed in Table 27.

TABLE 26

| Compound No | R¹ | R² | R³ | R⁴ | Xn | R⁸ |
|---|---|---|---|---|---|---|
| XII-5 | H | H | H | H | 4-Me | Me |
| XII-6 | H | H | H | H | 2-Cl | Me |
| XII-7 | H | H | H | H | 3-Cl | Me |
| XII-8 | H | H | H | H | 4-CF₃ | Me |
| XII-9 | H | H | H | H | 4-Ph | Me |
| XII-10 | H | H | H | H | 2,4-Cl₂ | Me |
| XII-11 | H | H | H | H | 3,4-Cl₂ | Me |

TABLE 27

| Compound No | Description | ¹H-NMR(CDCl₃) 400 MHz, δ |
|---|---|---|
| XII-5 | Brown oil | 0.77-0.81 (m, 1H), 0.91-0.94 (m, 1H), 1.10-1.14 (m, 1H), |
| | | 1.25-1.28 (m, 1H), 1.56-1.61 (m, 1H), 2.00-2.11 (m, 2H), |
| | | 2.31 (s, 3H), 2.47-2.51 (m, 1H), 3.06 (d, J = 13.8 Hz, 1H), |
| | | 3.24 (d, J = 13.8 Hz, 1H), 3.73 (s, 3H), 7.01 (d, J = 7.9 Hz, 2H), |
| | | 7.06 (d, J = 7.9 Hz, 2H). |
| XII-6 | Brown oil | 0.77-1.79 (m, 1H), 0.94-0.97 (m, 1H), 1.08-1.11 (m, 1H), |
| | | 1.29-1.33 (m, 1H), 1.56-1.60 (m, 1H), 2.01-2.19 (m, 2H), |
| | | 2.50-2.61 (m, 1H), 3.30 (d, J = 14.0 Hz, 1H), 3.56 (d, J = 14.0 Hz, |
| | | 1H), 3.75 (s, 3H), 7.15-7.36 (m, 4H). |
| XII-7 | Tan oil | 0.81 (ddd, J = 9.1, 7.2, 3.3 Hz, 1H), 0.96 (ddd, J = 9.1, 7.2, 3.5 Hz, |
| | | 1H), 1.13 (ddd, J = 9.9, 7.2, 3.3 Hz, 1H), 1.31 (ddd, J = 9.9, 7.2, |
| | | 3.5 Hz, 1H), 1.62 (ddd, J = 12.6, 7.6, 4.3 Hz, 1H), 1.98 (ddd, J = 13.2, |
| | | 8.6, 7.6 Hz, 1H), 2.13 (ddd, J = 12.6, 8.6, 7.6 Hz, 1H), |
| | | 2.50 (ddd, J = 13.2, 7.6, 4.3 Hz, 1H), 3.08 (d, J = 13.8 Hz, 1H), 3.25 (d, J = 13.8 Hz, |
| | | 1H), 3.74 (s, 1H), 7.0-7.1 (m, 1H), 7.13 (bs, 1H), |
| | | 7.2-7.3 (m, 2H). |
| XII-8 | Tan oil | 0.81 (ddd, J = 9.1, 7.2, 3.3 Hz, 1H), 0.97 (ddd, J = 9.1, 7.2, 3.7 Hz, |
| | | 1H), 1.11 (ddd, J = 9.9, 7.2, 3.3 Hz, 1H), 1.32 (ddd, J = 9.9, 7.2, |
| | | 3.7 Hz, 1H), 1.64 (ddd, J = 12.7, 7.6, 4.1 Hz, 1H), 1.97 (ddd, J = 13.0, |
| | | 8.9, 7.6 Hz, 1H), 2.15 (ddd, J = 12.7, 8.9, 7.6 Hz, 1H), |
| | | 2.50 (ddd, J = 13.0, 7.6, 4.1 Hz, 1H), 3.15 (d, J = 13.8 Hz, 1H), 3.33 (d, J = 13.8 Hz, |
| | | 1H), 3.74 (s, 3H), 7.26 (d, J = 8.4 Hz, 2H), 7.52 (d, J = 8.4 Hz, |
| | | 2H). |
| XII-9 | Pale yellow | 0.80 (ddd, J = 9.1, 7.2, 3.3 Hz, 1H), 0.95 (ddd, J = 9.1, 7.2, 3.5 Hz, |
| | oil | 1H), 1.14 (ddd, J = 9.9, 7.2, 3.3 Hz, 1H), 1.31 (ddd, J = 9.9, 7.2, |
| | | 3.5 Hz, 1H), 1.66 (dd, J = 12.1, 7.2 Hz, 1H), 2.0-2.2 (m, 2H), |
| | | 2.5-2.6 (m, 1H), 3.14 (d, J = 13.8 Hz, 1H), 3.32 (d, J = 13.8 Hz, 1H), |
| | | 3.75 (s, 3H), 7.21 (d, J = 8.4 Hz, 2H), 7.3-7.4 (m, 1H), 7.4-7.5 (m, |
| | | 2H), 7.49 (d, J = 8.4 Hz, 2H), 7.5-7.6 (m, 2H). |
| XII-10 | Brown oil | 0.79-0.82 (m, 1H), 0.95-0.99 (m, 1H), 1.08-1.11 (m, 1H), |
| | | 1.29-1.33 (m, 1H), 1.62-1.67 (m, 1H), 1.95-1.97 (m, 1H), |
| | | 2.12-2.19 (m, 1H), 2.50-2.58 (m, 1H), 3.27 (d, J = 14.2 Hz, 1H), |
| | | 3.51 (d, J = 14.2 Hz, 1H), 3.75 (s, 3H), 7.11-7.12 (m, 2H), |
| | | 7.36-7.38 (m, 1H) |
| XII-11 | Brown oil | 0.86-0.89 (m, 1H), 0.97-1.00 (m, 1H), 1.10-1.13 (m, 1H), |
| | | 1.31-1.35 (m, 1H), 1.58-1.68 (m, 1H), 1.92-2.00 (m, 1H), |
| | | 2.12-2.20 (m, 1H), 2.50-2.57 (m, 1H), 3.08 (d, J = 14.0 Hz, 1H), |
| | | 3.19 (d, J = 14.0 Hz, 1H), 3.73 (s, 3H), 6.99 (d, J = 8.0 Hz, 1H), |
| | | 7.25 (d, J = 7.6 Hz, 1H), 7.34 (d, J = 8.0 Hz, 1H) |

The followings are Formulation Examples and Experiment Examples, in which carriers (diluents) and auxiliary agents, as well as the mixing ratio thereof for active ingredients may vary within a wide range. “Parts” in each Formulation Example means “parts by weight”.

### Formulation Example 1

### Wettable Formulation

Compound (1-1) 50 parts

Lignin sulfonate 5 parts

Alkyl sulfonate 3 parts

Diatomaceous earth 42 parts

were ground and mixed to form a wettable formulation, which was used as being diluted in water.

### Formulation Example 2

### Powder Formulation

Compound (I-1) 3 parts

Clay 40 parts

Talc 57 parts

were ground and mixed, and used as a dusting formulation.

### Formulation Example 3

### Granule Formulation

Compound (I-1) 5 parts

Bentonite 43 parts

Clay 45 parts

Lignin sulfonate 7 parts

were mixed uniformly, combined with water and further kneaded, and subjected to an extruding granulator to obtain a granule, which was dried and used as a granule formulation.

### Formulation Example 4

### Emulsion Formulation

Compound (I-1) 20 parts

Polyoxyethylene alkylaryl ether 10 parts

Polyoxyethylene sorbitan monolaurate 3 parts

Xylene 67 parts

were mixed and dissolved uniformly to obtain an emulsion.

### Experimental Example 1

### Cucumber Gray Mold Preventing Effect Assay

To a cucumber (variety: SHARP1) plant in its cotyledon phase grown using a square plastic pot (6 cm-square) was used to cultivate, a wettable formulations such as Formulation Example 1 which was diluted and suspended in water at a certain concentration (500 mg/L) was sprayed at a rate of 1,000 L/ha. The sprayed leaves were air-dried, and loaded with a paper disc (8 mm in diameter) soaked with a spore suspension of a cucumber gray mold microorganism, and kept at 20 degrees C. and a high humidity. 4 Days after inoculation, the cucumber gray mold severity was investigated, and the protective value was calculated by the following equation.

TABLE 28

| Disease index | % Area of disease lesion |
|---|---|
| 0 | No disease |
| 0.5 | % Area of disease lesion < 10% |
| 1 | 10% ≦ % Area of disease lesion < 20% |
| 2 | 20% ≦ % Area of disease lesion < 40% |
| 3 | 40% ≦ % Area of disease lesion < 60% |
| 4 | 60% ≦ % Area of disease lesion < 80% |
| 5 | 80% ≦ % Area of disease lesion |

Protective value (%)=(1−mean disease index in sprayed plot/mean disease index in unsprayed plot)×100

In the assay described above, Compounds I-1, I-2, I-3, I-4, I-5, I-6, I-7, I-8, I-21, I-22, I-27, I-28, I-49, I-50, I-61, I-62, I-63, I-101, I-151, I-301, I-302, for example, exhibited protective values of 80% or higher.

In an analogous assay in which modification was made to a certain concentration (25 mg/l), comparison was made with Compound (A) in which a cyclopropyl ring was fused with a cyclopentane ring described in Patent Literature 13 (JP-A 11-80126), and it was revealed that the inventive Compound (I-1) exhibited a higher activity.

TABLE 29

| Compound | Controlling value |
|---|---|
| I-1 | 100% |
| Comparative Compound (A) | 54% |

[Chem. 35]

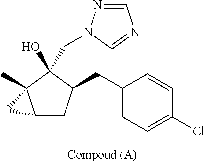

### Comparative Compound (A)

### 3-(4-chlorobenzyl)-1-methyl-2-(1H-1,2,4-triazol-1-ylmethyl)bicyclo[3.1.0]hexan-2-ol (Described in JP-A 11-80126)

### Experiment Example 2

### Wheat Leaf Rust Preventing Effect Assay

Onto a wheat plant (variety: NORIN No. 61) grown to the two-leaf phase using a square plastic pot (6 cm-square), a wettable formulations such as Formulation Example 1 which was diluted and suspended in water at a certain concentration (500 mg/L) was sprayed at a rate of 1,000 L/ha. The sprayed leaves were air-dried, and inoculated with wheat leaf rust microorganism's spore (adjusted at 200 spores/vision, Gramin S was added at 60 ppm) by spraying, and kept at 25 degrees C. and a high humidity for 48 hours. Thereafter, the plant was kept in a greenhouse. 9 to 14 days after inoculation, the wheat leaf rust severity was investigated, and the protective value was calculated by the following equation.

TABLE 30

| Disease index | Leaf rust damage scale by Peterson |
|---|---|
| 0 | No damage |
| 0.5 | Less than 1% |
| 1 | 1% or higher and less than 5% |
| 2 | 5% or higher and less than 10% |
| 3 | 10% or higher and less than 30% |
| 4 | 30% or higher and less than 50% |
| 5 | 50% or higher |

Protective value (%)=(1−mean disease index in sprayed plot/mean disease index in unsprayed plot)×100

In the assay described above, Compounds I-1, I-2, I-3, I-4, I-5, I-6, I-7, I-8, I-21, I-22, I-27, I-28, I-49, I-50, I-61, I-62, I-63, I-101, I-151, I-301, I-302 for example, exhibited protective values of 80% or higher.

### Experiment Example 3

### Wheat Powdery Mildew Preventing Effect Assay

Onto a wheat plant (variety: NORIN No. 61) grown to the two-leaf phase using a square plastic pot (6 cm-square), a wettable formulations such as Formulation Example 1 which was diluted and suspended in water at a certain concentration (500 mg/L) was sprayed at a rate of 1,000 L/ha. The sprayed leaves were air-dried, and splashed with wheat powdery mildew microorganism's spore, and thereafter kept in a greenhouse. 14 Days after inoculation, the wheat powdery mildew severity was investigated, and the protective value was calculated by the following equation.

TABLE 31

| Disease index | % Area of disease |
|---|---|
| 0 | No disease |
| 0.5 | % Area of disease of Less than 1% |
| 1 | % Area of disease of 1% or higher and less than 5% |
| 2 | % Area of disease of 5% or higher and less than 10% |
| 3 | % Area of disease of 10% or higher and less than 30% |
| 4 | % Area of disease of 30% or higher and less than 50% |
| 5 | % Area of disease of 50% or higher |

Protective value (%)=(1−mean disease index in sprayed plot/mean disease index in unsprayed plot)×100

In the assay described above, Compounds I-1, I-2, I-3, I-4, I-5, I-6, I-7, I-8, I-21, I-22, I-27, I-28, I-49, I-50, I-61, I-62, I-63, I-101, I-151, I-301, I-302, for example, exhibited protective values of 80% or higher.

### Experiment Example 4

### Assay for Wheat Powdery Mildew Preventing Effect by Seed Treatment

2 mg of a test compound was dissolved in 18 microliter of DMSO, and applied to 1 g of wheat seeds in a vial. On the next day, 10 seeds/pot were seeded to 1/10000 a pots, which were cultivated in a greenhouse with supplying water underneath. In the greenhouse, a diseased wheat seedling as an inoculant was placed, whereby keeping an infectious condition all the time. 7, 14, 28 and 56 days after seeding, severity was investigated by the following criteria, and the protective value was calculated by the following equation.

TABLE 32

| Disease index | % Area of disease |
|---|---|
| 0 | No disease |
| 0.5 | % Area of disease of Less than 1% |
| 1 | % Area of disease of 1% or higher and less than 5% |
| 2 | % Area of disease of 5% or higher and less than 10% |
| 3 | % Area of disease of 10% or higher and less than 30% |
| 4 | % Area of disease of 30% or higher and less than 50% |
| 5 | % Area of disease of 50% or higher |

Protective value (%)=(1−mean disease index in sprayed plot/mean disease index in unsprayed plot)×100

### <Powdery Mildew Controlling Index>

1: Protective value of 0 to 20

2: Protective value of 21 to 40

3: Protective value of 41 to 60

4: Protective value of 61 to 80

5: Protective value of 81 to 100

In the assay described above, Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited, in the seed treatment test, controlling index of 4 or higher against the wheat powdery mildew in stems and leaves.

### Experiment Example 5

### Assay for Antimicroorganism Effect on Various Pathogenic Microorganism and Hazardous Microorganisms

In this Experiment Example, a method described below was employed to test the antimicroorganism effects of inventive compounds on various pathogenic molds for plants and hazardous microorganism for industrial materials.

### <Testing Methods>

10 mg of each of inventive compounds was weighed and dissolved in 2 ml of dimethyl sulfoxide. 0.6 ml of this solution was added to 60 ml of a PDA medium (potato dextrose agar medium) and at about 60 degrees C., which was mixed thoroughly in a 100-ml conical flask, and poured into a dish, where it was solidified, whereby obtaining a plate medium containing the inventive compound at the final concentration of 50 mg/l. On the other hand, a subject microorganism previously cultured on a plate medium was cut out using a cork borer whose diameter was 4 mm, and inoculated to the drug-containing plate medium described above. After inoculation, the dish was grown at the optimum growth temperature for respective microorganism (for this growth temperature, see, for example, LIST OF CULTURES 1996 microorganisms 10th edition, Institute for Fermentation (foundation)) for 1 to 3 days, and the microorganism growth was measured as a diameter of its flora. The growth degree of the microorganism on the drug-containing plate medium thus obtained was compared with the growth degree of the microorganism in the untreated group, and % mycelial extension inhibition was calculated by the following equation.

R=100(dc−dt)/dc

wherein R=% mycelial extension inhibition, dc=flora diameter in untreated plate, dt=flora diameter in treated plate.

The results obtained as above were evaluated as one of the 5 grades according to the following criteria.

### <Growth Inhibition Grade>

5: % Mycerial extension inhibition of 90% or higher

4: % Mycerial extension inhibition of less than 90 to 70% or higher

3: % Mycerial extension inhibition of less than 70 to 40% or higher

2: % Mycerial extension inhibition of less than 40 to 20% or higher

1: % Mycerial extension inhibition of less than 20%

The results of the evaluation of the assays described above were shown below.

Against wheat septoria bloth microorganism (Septoria tritici), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against wheat stagonospora blotch microorganism (Phaeosphaeria nodorum), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against wheat eye spot microorganism (Pseudocercosporella herpotrichoides), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against wheat pink snow mold microorganism (Microdochium nivale), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against wheat take-all microorganism (Gaeumannomyces graminis), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against barley stripe microorganism (Pyrenophora graminea), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against barley scald microorganism (Rhynchosporium secalis), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against wheat fusarium blight microorganism (Fusarium graminearum), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against barley loose smut microorganism (Ustilago nuda), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against rice blast microorganism (Pyricularia oryzae), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against ricesheath blight microorganism (Rhizoctonia solani), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against rice bakanae disease microorganism (Giberella fujikuroi), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against rice seedling blight microorganism (Rhizopus) (Rhizopus oryzae), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against apple alternaria blotch microorganism (Alternaria alternata), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against sclerotinia rot microorganism (Sclerotinia sclerotiorum), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against gray mold microorganism (Botritis cinerea), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against anthracnose microorganism (Glomerella cingulata), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against cucumber fusarium wilt microorganism (Fusarium oxysporum), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against citrous blue mold microorganism (Penicillium italicum), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against sugar beet cercospora leaf spot microorganism (Cercospora beticola), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against a microorganism deteriorating paper, pulp, fiber, leather, paint and the like, namely, Aspergillus microorganism (Aspergillus sp.), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against a microorganism deteriorating paper, pulp, fiber, leather, paint and the like, namely, Tricoderma microorganism (Trichoderma sp.), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against a microorganism deteriorating paper, pulp, fiber, leather, paint and the like, namely, penicillium microorganism (Penicillium sp.), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against a microorganism deteriorating paper, pulp, fiber, leather, paint and the like, namely, Cladosporium microorganism (Cladosporium sp.), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against a microorganism deteriorating paper, pulp, fiber, leather, paint and the like, namely, Mucor microorganism (Mucor sp.), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against a microorganism deteriorating paper, pulp, fiber, leather, paint and the like, namely, Aureobasidium microorganism (Aureobasidium sp.), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against a microorganism deteriorating paper, pulp, fiber, leather, paint and the like, namely, Curvularia microorganism (Curvularia sp.), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 4.

Against a wood denaturing microorganism (Tyromyces palustris), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

Against a wood denaturing microorganism (Coriolus versicolor), Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth inhibition grades as high as 5.

### Experiment Example 6

### Rice Growth Regulation Assay

36 mg of a test compound was dissolved in 3.6 ml of DMSO, and applied to 180 g of rice seeds in a vial. After soaking the seeds and promoting germination, the seeds were seeded to seedling boxes at a rate of 180 g/box, allowed to germinate in the seedling boxes, and then cultivated in a greenhouse at 35 degrees C. 20 Days after seeding, the plant height of the seedlings in each treatment group was surveyed in 10 locations, and the % plant height suppression was calculated by the following Equation 6.

R=100(hc−ht)/hc

Wherein R=% Plant height suppression, hc=mean untreated plant height, ht=mean treated plant height.

The results obtained above were assigned to one of the following 5 grades of the growth regulation.

### <Growth Regulation Grade>

5: % Plant height suppression of 50% or higher

4: % Plant height suppression of less than 50 to 30% or higher

3: % Plant height suppression of less than 30 to 20% or higher

2: % Plant height suppression of less than 20 to 10% or higher

1: % Plant height suppression of 10% or less

In the assay described above, Compounds I-1, I-2, I-3, I-4, I-5, I-6, I-7, I-8, I-21, I-22, I-27, I-28, I-49, I-50, I-61, I-62, I-63, I-64, I-101, I-151, I-301, I-302 exhibited growth regulation grades of 4 or higher in the growth of rice plant.

### INDUSTRIAL APPLICABILITY

A 5-benzyl-4-azolylmethyl-4-spiro[2.4]heptanol derivative represented by Formula (I) according to the invention is not only useful as an active ingredient of an agro-horticultural fungicide, but also useful as a plant growth regulator which regulates the growth of a variety of crops and horticultural plants whereby exhibiting yield-increasing effects or quality-improving effects, as well as an industrial material protecting agent which protects an industrial material from a wide range of hazardous microorganisms which invades such materials.

1. A 5-benzyl-4-azolylmethyl-4-spiro[2.4]heptanol derivative represented by Formula (I): 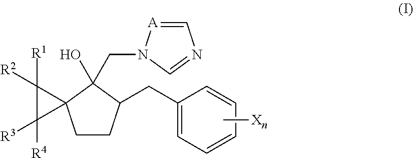
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and A denotes a nitrogen atom or a methyne group.

2. A method for producing a 5-benzyl-4-azolylmethyl-4-spiro[2.4]heptanol derivative represented by Formula (I) comprising reacting an oxirane derivative represented by Formula (II), which is obtained by oxiranylating a carbonyl compound represented by Formula (IV), with a 1,2,4-triazole or imidazole compound represented by Formula (III): 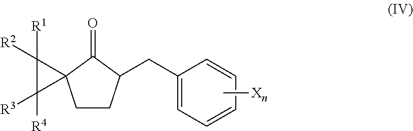
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; 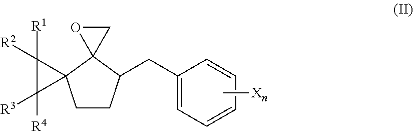
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; 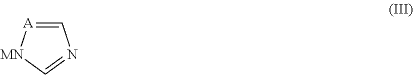
wherein M denotes a hydrogen atom or an alkaline metal; and A denotes a nitrogen atom or a methyne group; 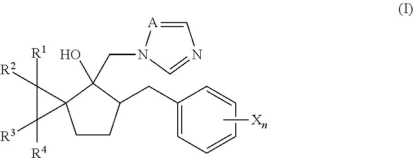
wherein X, N, R¹, R², R³ and R⁴ correspond to the X, N, R¹, R², R³ and R⁴ as defined in Formula II described above; and A corresponds to the A as defined in Formula III described above.

3. An agro-horticultural agent and an industrial material protecting agent containing a 5-benzyl-4-azolylmethyl-4-spiro[2.4]heptanol derivative according to claim 1 represented by Formula (I): 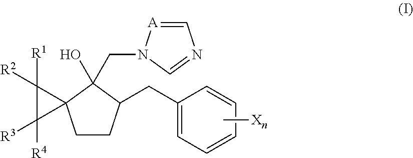
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and A denotes a nitrogen atom or a methyne group.

4. An oxirane derivative represented by Formula (II) as an intermediate compound for producing the 5-benzyl-4-azolylmethyl-4-spiro[2.4]heptanol derivative according to claim 1: 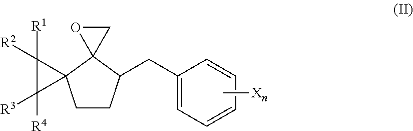
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group.

5. A carbonyl compound represented by Formula (IV) as an intermediate compound for producing the 5-benzyl-4-azolylmethyl-4-spiro[2.4]heptanol derivative according to claim 1: 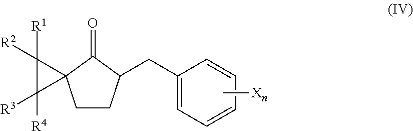
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group.

6. A 2-(2-haloethyl)cyclopentanone compound represented by Formula (Va) as an intermediate compound for producing the 5-benzyl-4-azolylmethyl-4-spiro[2.4]heptanol derivative according to claim 1: 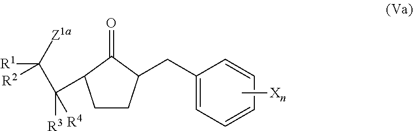
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same or different; R¹, R², R³, R⁴ each independently denotes a hydrogen atom, a halogen atom or a C₁-C₅ alkyl group; and Z^{1a} is a halogen atom other than a fluorine atom.

7. A 2-(lower alkoxy)alkyl ketoester compound represented by Formula (XVIII) as an intermediate compound for producing the 5-benzyl-4-azolylmethyl-4-spiro[2.4]heptanol derivative according to claim 1: 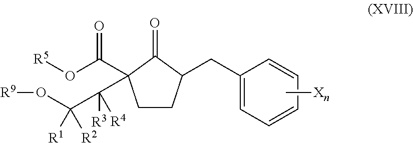
wherein X denotes a halogen atom, a C₁-C₅ alkyl group, a C₁-C₅ haloalkyl group, a C₁-C₅ alkoxy group, a C₁-C₅ haloalkoxy group, a phenyl group, a cyano group or a nitro group; n denotes an integer of 0 to 5; when n is not less than 2, Xs may be the same of different; R¹, R², R³, R⁴ each independently denotes a hydrogen atom, a halogen atom or a C₁-C₅ alkyl group; and R⁵ and R⁹ each independently denotes a C₁-C₄ lower alkyl group.
